SEQUENCE LISTING (35 sequences)

SEQ ID NO: 1 - DNA, 6 bp, Homo sapiens
gvagaw

SEQ ID NO: 2 - DNA, 12 bp, Artificial Sequence
  DNA sequence that transcribes a candidate RNA motif
gaagaagaagat

SEQ ID NO: 3 - RNA, 21 nt, Artificial Sequence
  shRNA molecule targeting PD-L1
gaccuauaugugguagaguau

SEQ ID NO: 4 - RNA, 21 nt, Artificial Sequence
  shRNA molecule targeting PD-L1
cgaauuacugugaaagucaau

SEQ ID NO: 5 - DNA, 21 bp, Artificial Sequence
  nucleic acid encoding an shRNA molecule targeting PD-L1
gacctatatgtggtagagtat

SEQ ID NO: 6 - DNA, 21 bp, Artificial Sequence
  nucleic acid encoding an shRNA molecule targeting PD-L1
cgaattactgtgaaagtcaat

SEQ ID NO: 7 - DNA, 21 bp, Artificial Sequence
  nucleic acid encoding an shRNA molecule targeting HuR
cgtggatcagactacaggttt

SEQ ID NO: 8 - DNA, 21 bp, Artificial Sequence
  nucleic acid encoding an shRNA molecule targeting HuR
accatgacaaactatgaagaa

SEQ ID NO: 9 - DNA, 22 bp, Artificial Sequence
  synthetic oligonucleotide primer
gcaggaggagaaccatacagac

SEQ ID NO: 10 - DNA, 20 bp, Artificial Sequence
  synthetic oligonucleotide primer
atgatttcggctgatcgact

SEQ ID NO: 11 - DNA, 22 bp, Artificial Sequence
  synthetic oligonucleotide primer
ccaacctgagtcaaacagatga

SEQ ID NO: 12 - DNA, 22 bp, Artificial Sequence
  synthetic oligonucleotide primer
aagtaataccatcccccgactt

SEQ ID NO: 13 - DNA, 22 bp, Artificial Sequence
  synthetic oligonucleotide primer
tgaaagcagttgagtccaagaa

SEQ ID NO: 14 - DNA, 22 bp, Artificial Sequence
  synthetic oligonucleotide primer
aaagacaactgcggaactcaat

SEQ ID NO: 15 - DNA, 22 bp, Artificial Sequence
  synthetic oligonucleotide primer
caatgttagaaaaaggccaagg

SEQ ID NO: 16 - DNA, 22 bp, Artificial Sequence
  synthetic oligonucleotide primer
gttcacgtttcttcccaatttc

SEQ ID NO: 17 - DNA, 22 bp, Artificial Sequence
  synthetic oligonucleotide primer
gcggtgattttcatgactacaa

SEQ ID NO: 18 - DNA, 22 bp, Artificial Sequence
  synthetic oligonucleotide primer
aaatcccatgtatctgcttgct

SEQ ID NO: 19 - DNA, 22 bp, Artificial Sequence
  synthetic oligonucleotide primer
tgcagtgttctacaccttgctt

SEQ ID NO: 20 - DNA, 22 bp, Artificial Sequence
  synthetic oligonucleotide primer
actgaagccattttgtttggat

SEQ ID NO: 21 - DNA, 22 bp, Artificial Sequence
  synthetic oligonucleotide primer
cggagtcaacggatttggtcgt

SEQ ID NO: 22 - DNA, 20 bp, Artificial Sequence
  synthetic oligonucleotide primer
tctcagccttgacggtgcca

SEQ ID NO: 23 - DNA, 22 bp, Artificial Sequence
  synthetic oligonucleotide primer
atggaaggtaaagaacctgcaa

SEQ ID NO: 24 - DNA, 22 bp, Artificial Sequence
  synthetic oligonucleotide primer
tggaaggctaggattgacaaat

SEQ ID NO: 25 - DNA, 21 bp, Artificial Sequence
  synthetic oligonucleotide primer
gacctgcaaagagcaatgaag

SEQ ID NO: 26 - DNA, 21 bp, Artificial Sequence
  synthetic oligonucleotide primer
acccctcttcaactcccacta

SEQ ID NO: 27 - DNA, 20 bp, Artificial Sequence
  synthetic oligonucleotide primer
gtggtaaaacccctgggaat

SEQ ID NO: 28 - DNA, 20 bp, Artificial Sequence
  synthetic oligonucleotide primer
aggatagcacgagctggaaa

SEQ ID NO: 29 - DNA, 20 bp, Artificial Sequence
  synthetic oligonucleotide primer
tttgcttgaggctgatcctt

SEQ ID NO: 30 - DNA, 20 bp, Artificial Sequence
  synthetic oligonucleotide primer
gattgactctgcagccaaca

SEQ ID NO: 31 - protein, 10 aa, Artificial Sequence
  peptide that blocks the PD-L1 binding site for NBS1 RNA
KKCGIQDTNS

SEQ ID NO: 32 - protein, 8 aa, Artificial Sequence
  cell penetrating peptide
RRRRRRRR

SEQ ID NO: 33 - protein, 12 aa, Artificial Sequence
  cell penetrating peptide
GRKKRRQRRRPQ

SEQ ID NO: 34 - protein, 16 aa, Artificial Sequence
  cell penetrating peptide
RQIKIWFQNRRMKWKK

SEQ ID NO: 35 - protein, 29 aa, Artificial Sequence
  cell penetrating peptide
IAWVKAFIRKLRKGPLGGPLGIAGQRGDS

(19) United States
(12) Patent Application Publication
(10) Pub. No.: US 2020/0338112 A1
(43) Pub. Date: Oct. 29, 2020

(54) METHODS AND MATERIALS FOR TREATING CANCER

(71) Applicant: Mayo Foundation for Medical Education and Research, Rochester, MN (US)

(72) Inventors: Zhenkun Lou, Rochester, MN (US); Haidong Dong, Rochester, MN (US); Xinyi Tu, Rochester, MN (US)

(73) Assignee: Mayo Foundation for Medical Education and Research, Rochester, MN (US)

(21) Appl. No.: 16/959,176

(22) PCT Filed: Jan. 4, 2019
(86) PCT No.: PCT/US2019/012293

§ 371 Date: Jun. 30, 2020

Related U.S. Application Data
(60) Provisional application No. 62/613,506, filed on Jan. 4, 2018.

(51) Int. Cl. A61K 31/7105; A61K 38/08; A61K 33/243; C12N 15/113

(52) U.S. Cl. CPC A61K 31/7105; A61K 38/08; A61K 45/06; C12N 15/113; A61K 33/243

(57) ABSTRACT

This document relates to methods and materials for treating cancer. For example, methods and materials for inhibiting the function of an intracellular domain of programmed death-ligand 1 (PD-L1) to sensitize cancer cells in a mammal (e.g., a human) to one or more cancer treatments (e.g., radiation therapy and/or chemotherapy) are provided. Also provided are methods and materials for identifying compounds that inhibit the function of an intracellular PD-L1 domain.

### CROSS-REFERENCE TO RELATED APPLICATIONS

This application claims the benefit of U.S. Patent Application Ser. No. 62/613,506, filed on Jan. 4, 2018. The disclosure of the prior application is considered part of (and is incorporated by reference in) the disclosure of this application.

### BACKGROUND

### 1. Technical Field

This document relates to methods and materials for treating cancer. For example, this document provides methods and materials for inhibiting the function of an intracellular domain of programmed death-ligand 1 (PD-L1) to sensitize cancer cells in a mammal (e.g., a human) to one or more cancer treatments (e.g., radiation therapy and/or chemotherapy). This document also provides methods and materials for identifying compounds that inhibit the function of an intracellular PD-L1 domain.

### 2. Background Information

PD-L1, also called B7-H1, is an immune checkpoint protein that regulates the immune system through its binding of the programmed cell death protein 1 (PD-1) receptor. In the tumor microenvironment, overexpression of PD-L1 on tumor cells helps suppress antitumor immunity (Dong et al., Nat Med. 8:793-800, (2002); Hamanishi et al., Int. J. Clin. Oncol. 21:462-473 (2016); Dong et al., Nat. Med. 5:1365-1369 (1999); Chen et al., J. Clin. Invest. 125:3384-3391 (2015); He et al., Sci. Rep. 5:13110 (2015); Chen et al., Clin. Cancer Res. 18:6580-6587 (2012); Ohaegbulam et al., Trends Mol. Med. 21:24-33 (2015); and Postow et al., J. Clin. Oncol. 33:1974-1982 (2015)).

### SUMMARY

This document provides methods and materials for treating cancer. For example, a compound that inhibits the function of an intracellular domain of PD-L1 can be administered to a mammal (e.g., a human) having cancer to sensitize cancer cells in the mammal to one or more cancer treatments. In some cases, a compound that inhibits the function of an intracellular domain of PD-L1 can be administered to a mammal (e.g., a human) having cancer together with one or more cancer treatments to reduce the severity of the cancer and/or to reduce a symptom of the cancer. This document also provides methods and materials for identifying compounds that inhibit the function of an intracellular PD-L1 domain. For example, a candidate compound can be contacted with PD-L1 (e.g., one or more cells expressing PD-L1), and the ability of the candidate compound to inhibit the function of an intracellular domain of PD-L1 can be determined. In some cases, a compound identified herein (e.g., a compound that inhibits the function of an intracellular domain of PD-L1) can be used to sensitize cancer cells to one or more cancer treatments as described herein. In some cases, a compound identified herein (e.g., a compound that inhibits the function of an intracellular domain of PD-L1) can be used to treat a mammal having cancer as described herein.

As described herein, an intracellular domain of PD-L1 has intrinsic functions that are independent of its established role as a PD1 ligand. For example, PD-L1 is important for proper DNA damage response (DDR) and DNA repair, and regulates the expression of multiple DNA damage response factors by affecting their mRNA stability. The PD-L1 intracellular domain can carry out these functions by binding with NBS1 mRNA and/or by interacting with the RNA binding protein HuR. As demonstrated herein, knockdown of PD-L1 sensitized cancer cells to cisplatin and ionizing radiation (IR). Having the ability to inhibit the function of a PD-L1 intracellular domain can allow clinicians to sensitize cancer cells to cytotoxic cancer treatments such as radiation therapies and chemotherapies.

In general, one aspect of this document features a method for treating a mammal having cancer. The method includes, or consists essentially of, administering a compound that inhibits the function of an intracellular domain of PD-L1 to the mammal under conditions wherein cancer cells present are sensitized to one or more cancer treatments. The mammal can be a human. The cancer can be breast cancer. The cancer can be colorectal cancer. The one or more cancer treatments can include radiation therapy, chemotherapy, hormone therapy, targeted therapy, and/or cytotoxic therapy. In some cases, the one or more cancer treatments can include radiation therapy (e.g., irradiation). In some cases, the one or more cancer treatments can include chemotherapy (e.g., cisplatin). The compound that inhibits the function of an intracellular domain of PD-L1 can include one or more shRNA molecules. The one or more shRNA molecules can be encoded by a nucleic acid comprising the sequence GACCTATATGTGGTAGAGTAT (SEQ ID NO:5). The one or more shRNA molecules can include the sequence GACCUAUAUGUGGUAGAGUAU (SEQ ID NO:3). The one or more shRNA molecules can be encoded by a nucleic acid comprising the nucleic acid sequence CGAATTACTGTGAAAGTCAAT (SEQ ID NO:6). The one or more shRNA molecules can include the sequence CGAAUUACUGUGAAAGUCAAU (SEQ ID NO:4). The compound that inhibits the function of an intracellular domain of PD-L1 can include one or more polypeptides. The one or more polypeptides can have at least 20% identity to the sequence KKCGIQDTNS (SEQ ID NO:31). The one or more polypeptides can include the sequence KKCGIQDTNS (SEQ ID NO:31). The one or more polypeptides also can include a cell penetrating peptide (CPP). The CPP can include the sequence RRRRRRRR (SEQ ID NO:32)

In another aspect, this document features a method for treating a mammal having cancer. The method includes, or consists essentially of, administering a compound that inhibits the function of an intracellular domain of PD-L1 to the mammal; and administering one or more cancer treatments the mammal under conditions wherein number of cancer cells present in the mammal is reduced. The mammal can be a human. The cancer can be breast cancer. The cancer can be colorectal cancer. The one or more cancer treatments can include radiation therapy, chemotherapy, hormone therapy, targeted therapy, and/or cytotoxic therapy. In some cases, the one or more cancer treatments can include radiation therapy (e.g., irradiation). In some cases, the one or more cancer treatments can include chemotherapy (e.g., cisplatin). The compound that inhibits the function of an intracellular domain of PD-L1 can include one or more shRNA molecules. The one or more shRNA molecules can be encoded by a nucleic acid comprising the sequence GACCTATATGTGGTAGAGTAT (SEQ ID NO:5). The one or more shRNA molecules can include the sequence GACCUAUAUGUGGUAGAGUAU (SEQ ID NO:3). The one or more shRNA molecules can be encoded by a nucleic acid comprising the nucleic acid sequence CGAATTACTGTGAAAGTCAAT (SEQ ID NO:6). The one or more shRNA molecules can include the sequence CGAAUUACUGUGAAAGUCAAU (SEQ ID NO:4).

In another aspect, this document features a method for identifying a compound that inhibits the function of an intracellular PD-L1 domain. The method includes, or consists essentially of, contacting a candidate compound with an intracellular domain of PD-L1, determining if the candidate compound inhibits the function of the intracellular domain of PD-L1, and identifying the candidate compound as a compound that inhibits the function of the intracellular PD-L1 domain when the function of the intracellular PD-L1 domain is reduced or eliminated. The method can include contacting the candidate compound with cell-free PD-L1. The method can include contacting the candidate compound with cells expressing PD-L1. The PD-L1 can be endogenous PD-L1. The PD-L1 can be recombinant PD-L1. The determining step can include a co-immunoprecipitation. The determining step can include a nuclear run-on assay.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1a-1f show that PD-L1 is required for DNA damage response. FIG. 1a contains colony formation experiments of HCT116 cells under either ionizing radiation (IR) (0, 2, 4 Gy) or cisplatin (0, 0.5, 1 and 2 μM) treatment. Knockdown of PD-L1 significantly sensitized cells to IR and cisplatin, while restoring PD-L1 in knockdown cells rescued the phenomenon (±s.e.m., n=3). Lentiviral shRNAs targeting PD-L1 was used to knockdown PD-L1, while lentiviral shRNA which did not target any gene was used as a negative control. FIG. 1b contains knockdown and overexpression efficiency validation of PD-L1 in HCT116 using western blot; glyceraldehyde 3-phosphate dehydrogenase (GAPDH) was used as a loading control. FIG. 1c shows detection of PD-L1 depletion efficiency in U2OS cells; GAPDH was used as a loading control. FIG. 1d shows the kinetics of rH2AX foci under PD-L1 depletion in U2OS cells. The formation of rH2AX foci was sustained in PD-L1 depletion group compared to control group, data is represented as mean±s.e.m for each view. FIG. 1e contains an assessment of wild-type (+/+, n=16), PD-L1 heterozygous (+/−, n=24), and PD-L1 knockout (−/−, n=16) BALB/C mice that were exposed to whole body irradiation (7 Gy). Comparisons between groups were made using the Log-rank test. FIG. 1f shows that durvalumab (10 μg/mL, 24 hours before IR) did not sensitize cells to DNA damage stimulation. Result were based upon colony formation (±s.e.m., n=3).

FIGS. 2a-2f show that PD-L1 is involved in DNA damage response. FIG. 2a contains an assessment of MDA-MB-231 cells that were exposed to the indicated doses of IR or cisplatin. Cell survival was assessed using the MTS cell proliferation assay (±s.e.m., n=3). FIG. 2b contains a western blot analysis of PD-L1 expression in control and PD-L1 depleted MDA-MB-231 cells. GAPDH was used as a loading control. FIG. 2c shows that PD-1 is not expressed in HCT116 or MDA-MB-231 cells. JVM2 cells were used as a positive control of PD-1 expression. FIG. 2d shows that overexpression of PD-L1 renders HeLa cells resistant to both IR and cisplatin (±s.e.m., n=3). FIG. 2e shows validation of overexpression of PD-L1 in HeLa cells. GAPDH was used as a loading control. FIG. 2f contains representative pictures of immunofluorescence results of rH2AX foci in PD-L1 depleted U2OS cells.

FIGS. 3a-3k show that PD-L1 regulates NBS1 mRNA stability. FIGS. 3a and 3b contain western blot analyses of BRCA1, NBS1, RAD50, MRE11, and PD-L1 in HCT116 and MDA-MB-231 cells, respectively, that were infected with lentiviral control shRNA or two different shRNAs targeting PD-L1. GAPDH was used as a loading control. FIG. 3c contains a western blot of NBS1 protein levels in MDA-MB-231 cells infected with lentivirus control shRNA or two different shRNAs targeting PD-L1 were treated with DMSO (control) or MG132 (10 μM) for six hours before proteins were harvested. GAPDH and p53 were used as loading and positive control, respectively. FIG. 3d shows a quantification of NBS1 mRNA levels in HCT116 cells infected with lentiviral control shRNA, or two different shRNAs targeting PD-L1 with RT-PCR. GAPDH was used for normalization. FIGS. 3e, 3f, and 3g assess HCT116 cells treated with the transcription inhibitor actinomycin D (5 μg/mL). NBS1 mRNA levels were quantified using RT-PCR. Knockdown of PD-L1 in HCT116 decreased the half-life of NBS1 and BRCA1 mRNA, while restoring PD-L1 in the knockdown cells rescued the phenotype (±s.e.m., n=3). GAPDH was used for normalization. FIG. 3h shows a nuclear run on assay performed in MDA-MB-231 cells. The change of NBS1 and BRCA1 mRNA level was not due to transcription, since the transcription of BRCA1 was not changed, while the transcription of NBS1 was even increased after PD-L1 was knocked down (±s.e.m., n=3). GAPDH was used for normalization. FIG. 3i contains representative pictures of the subcellular localization of PD-L1. The immunofluorescence was performed in HCT116 cells. FIG. 3j shows the result of cross-linked RIP in MDA-MB-231. NBS1 was significantly enriched by PD-L1 compared to IgG. The result was shown as the percentage of input (±s.e.m., n=3, two tails t-test). FIG. 3k contains a schematic representation of the structure of PD-L1 and PD-L1 truncations. PD-L1 has three domains: an extracellular domain including the signal peptide, a transmembrane domain, and a cytoplasmic domain. Two truncations were constructed. The first truncation contained the extracellular domain only (E), and the second truncation contained both the transmembrane domain plus cytoplasmic domain (T+C).

FIGS. 4a-4h show that PD-L1 interacts with and stabilizes NBS1 mRNA. FIG. 4a shows a quantification of NBS1 and BRCA1 mRNA levels in MDA-MB-231 cells infected with lentiviral control shRNA, and two different lentiviral shRNAs targeting PD-L1 using real-time (RT)-PCR. GAPDH was used for normalization. FIGS. 4b and 4c assess MDA-MB-231 cells treated with the transcription inhibitor actinomycin D (5 μg/mL). NBS1 mRNA levels were quantified using qRT-PCR (±s.e.m., n=3). GAPDH was used for normalization. Knockdown of PD-L1 decreased the half-life of NBS1 mRNA in MDA-MB-231. After restoration of PD-L1 expression in PD-L1 knockdown MDA-MB-231 cells, the shorter half-life of NBS1 mRNA was rescued (±s.e.m., n=3). FIG. 4d shows that, after restoration of PD-L1 back into PD-L1 knockdown cells, the protein level of NBS1 was rescued. GAPDH was used as a loading control. FIG. 4e shows that NBS1 mRNA was significantly enriched by PD-L1 compared to the negative control IgG using RNA immunoprecipitation (RIP) assay. The result was shown as the percentage of input (±s.e.m., n=3). FIG. 4f contains an RNA pull down assay using in vitro biotin labeled NBS1 RNA in MDA-MB-231 cells. The binding affinity of the transmembrane and cytoplasmic (T+C) domain with NBS1 mRNA is the same as full length PD-L1, while the extracellular domain (E) alone cannot bind NBS1 mRNA. FIGS. 4g and 4h show that T+C domain and full length PD-L1 rescued both the protein and RNA level (±s.e.m., n=3, t-test, two tails, ***p value<0.001) of NBS1 in MDA-MB-231 cells depleted of PD-L1.

FIGS. 5a-5j show that PD-L1 collaborates with HuR to regulates NBS1 mRNA stability. FIGS. 5a and 5b contain results of an endogenous co-IP using HuR or PD-L1 antibodies in MDA-MB-231 cells. Both results showed that PD-L1 interacts with HuR. FIGS. 5c and 5d map the interaction between PD-L1 and HuR. The results showed that transmembrane and cytoplasmic domains of PD-L1 are important for binding with HuR, and HuR binds PD-L1 through its RRM3 domain. FIGS. 5e and 5f show that double knockdown of PD-L1 and HuR showed similar result as single knockdown. FIGS. 5g and 5h show that the decreased NBS1 protein and RNA level by PD-L1 depletion could be rescued by ectopic expression of HuR. GAPDH was used as a negative control. FIGS. 5i-5j show that knockdown of PD-L1 decreased the binding affinity of HuR with NBS1 and BRCA1 RNA, while knockdown of HuR also decreased the binding affinity of PD-L1 with NBS1 and BRCA1 RNA. The data was obtained from native RIP assay. The result was shown as the percentage of input (±s.e.m., n=3, two tails t-test).

FIGS. 6a-6i show that HuR interacts with PD-L1 and regulates NBS1 RNA stability. FIG. 6a contains a schematic representation of the structure of HuR and HuR truncations. FIG. 6b shows a native RIP assay using HuR antibody with detection of NBS1 mRNA by qRT-PCR in MDA-MB-231 cells. NBS1 mRNA was significantly enriched by HuR compared to control IgG. The result was shown as the percentage of input (±s.e.m., n=3). FIG. 6c shows an RNA pull down assay using in vitro biotin labeled NBS1 RNA in MDA-MB-231 cells, and the results suggested that all three RRM domains of HuR interact with NBS1 mRNA. FIG. 6d shows that HuR binds with the 3′ UTR of NBS1 RNA. The experiment was performed using a crosslinked RIP assay, and the measurement of different domain of NBS1 RNA was performed through qRT-PCR. The result was shown as the percentage of input (±s.e.m., n=3). FIGS. 6e and 6f show that knockdown of HuR significantly decreased both RNA (±s.e.m., n=3) using qRT-PCR and protein level of NBS1 using western blot in MDA-MB-231 cells. FIG. 6g shows that knockdown of HuR decreased the half-life of NBS1 mRNA in MDA-MB-231 cells (±s.e.m., n=3). Cells were harvested after the indicated time, and the result was obtained by qRT-PCR. FIG. 6h shows a validation of HuR knockdown and overexpression efficiency of the HuR rescue assay. GAPDH was used as a loading control. FIG. 6i shows a RIP assay using MDA-MB-231 cells. While both NBS1 and BRCA1 were significantly enriched by PD-L1 compared to negative control, only RRM2+3 and full length HuR rescued the lower affinity of PD-L1 with RNAs caused by HuR knockdown.

FIGS. 7a-7h show that PD-L1 regulates RNA stability genome-wide. FIG. 7a contains a heatmap of DNA damage related genes enriched by PD-L1 through RIP-seq. The map was plotted according to the z-score of log (number of peaks). FIG. 7b contains a heatmap of DNA damage genes enriched by PD-L1 through RNA seq. The map was plotted according to the z-score of log (normalized counts). FIG. 7c contains Venn diagrams of overlapping genes for RIP-seq and RNA-seq. The genes enriched by PD-L1 through RIP were significantly overlapping with the downregulated genes caused by PD-L1 knockdown. FIG. 7d shows that the overlapping genes of RIP-seq and RNA-seq were significantly enriched in multiple important biological including DNA damage related pathways. The data were obtained via a Gene Ontology (GO) analysis and shown as −log (p-value). FIG. 7e shows a validation of several DNA damage related genes enriched by both RIP-Seq and RNA-seq. GAPDH was used for normalization. FIG. 7f shows the top PD-L1 binding RNA motif (GVAGAW where V is A, C, or G, and where W is A or U; SEQ ID NO:1) identified using MEME ChIP software. FIG. 7g shows a dual luciferase reporter assay using pmirGLO vector. Two copies a DNA sequence that transcribes a candidate RNA motif (GAAGAAGAAGAT; SEQ ID NO:2) were inserted into the 3′UTR of firefly luciferase gene, then both empty vector and vector with the insert were transfected into control and PD-L1 knockdown cells to measure the signal of firefly luciferase. Renilla luciferase acted as an internal control. The data showed that the RNA motif increased the signal of percent firefly luciferase activity: renilla luciferase activity, while knockdown of PD-L1 significantly diminished this effect (±s.e.m., n=6, two tails t-test). FIG. 7h contains a model of the proposed role of PD-L1/HuR in RNA stability regulation. Top: In the presence of PD-L1, PD-L1 and HuR bind with targeted RNAs to enhance the latter's stability. As certain targeted RNAs are coding genes for DNA damage repair, cancer cells gain their resistance to DNA damage stimulation. Bottom: In the absence of PD-L1, the binding affinity of HuR with targeted RNAs compromises and leads to reduction of these targeted RNAs, and eventually cancer cells loss their resistance to DNA damage reagents.

FIGS. 8a and 8b show the design of a polypeptide that blocks the PD-L1 binding site for NBS1 RNA. FIG. 8a contains a schematic representation of intracellular (transmembrane plus cytoplasmic domain, T+C) PD-L1 truncations. FIG. 8b contains an image of an RNA pull down assay in MDA-MB-231 cells expressing different intracellular PD-L1 truncations using in vitro biotin labeled 3′ NBS1 RNA. These results indicate that targeting the intracellular regions (especially 270-279 aa) of PD-L1 with negative dominant peptides or small molecules can disrupt the interaction of PD-L1 with targeted (NBS1) RNAs in cancer cells.

### DETAILED DESCRIPTION

This document provides methods and materials for treating cancer. For example, a compound that inhibits (e.g., reduces or eliminates) the function of an intracellular domain of PD-L1 can be administered to a mammal (e.g., a human) having cancer (e.g., breast cancer or colorectal cancer) to sensitize cancer cells in the mammal to one or more cancer treatments (e.g., radiation therapy and/or chemotherapy). In some cases, one or more compounds that inhibit the function of an intracellular domain of PD-L1 can be administered to a mammal having cancer together with one or more cancer treatments to reduce the severity of the cancer, to reduce a symptom of the cancer, and/or to reduce the number of cancer cells present within the mammal.

Any appropriate mammal having cancer can be treated as described herein. For example, humans and other primates such as monkeys having cancer can be treated with one or more compounds that inhibit the function of an intracellular domain of PD-L1 and, optionally, can be treated with one or more cancer treatments to reduce the severity of the cancer, to reduce a symptom of the cancer, and/or to reduce the number of cancer cells present within the mammal within the human or other primate. In some cases, dogs, cats, horses, cows, pigs, sheep, mice, and rats having cancer can be treated with one or more compounds that inhibit the function of an intracellular domain of PD-L1, and, optionally, can be treated with one or more cancer treatments as described herein.

When treating a mammal (e.g., a human) having a cancer as described herein, the cancer can be any appropriate cancer. Examples of cancers that can be treated as described herein include, without limitation, breast cancer, colorectal cancer, kidney cancer, lung cancer, ovarian cancer, and melanoma.

Any appropriate method can be used to identify a mammal having cancer. For example, imaging techniques and biopsy techniques can be used to identify mammals (e.g., humans) having cancer.

Once identified as having a cancer (e.g., breast cancer or colorectal cancer), a mammal can be administered one or more compounds that inhibit (e.g., reduces or eliminates) the function of an intracellular domain of PD-L1, and, optionally, can be treated with one or more cancer treatments. The one or more cancer treatments can include any appropriate cancer treatments. In some cases, a cancer treatment can include surgery. In some cases, a cancer treatment can include radiation therapy. In some cases, a cancer treatment can include administration of a pharmacotherapy such as a chemotherapy, hormone therapy, targeted therapy, and/or cytotoxic therapy. Examples of cancer treatments include, without limitation, administration of one or more platinum compounds (e.g., a cisplatin or carboplatin), administration of one or more taxanes (e.g., paclitaxel, docetaxel, or an albumin bound paclitaxel such as nab-paclitaxel), administration of altretamine, administration of capecitabine, administration of cyclophosphamide, administration of etoposide (vp-16), administration of gemcitabine, administration of ifosfamide, administration of irinotecan (cpt-11), administration of liposomal doxorubicin, administration of melphalan, administration of pemetrexed, administration of topotecan, administration of vinorelbine, administration of one or more luteinizing-hormone-releasing hormone (LHRH) agonists (such as goserelin and leuprolide), administration of one or more anti-estrogen therapies (such as tamoxifen), administration of one or more aromatase inhibitors (such as letrozole, anastrozole, and exemestane), administration of one or more angiogenesis inhibitors (such as bevacizumab), administration of one or more poly(ADP)-ribose polymerase (PARP) inhibitors (such as olaparib, rucaparib, and niraparib), administration of external beam radiation therapy, administration of brachytherapy, administration of radioactive phosphorus, and administration of any combinations thereof. For example, a mammal having cancer can be administered one or more compounds that inhibit the function of an intracellular domain of PD-L1 and administered IR therapy. In another example, a mammal having cancer can be administered one or more compounds that inhibit the function of an intracellular domain of PD-L1 and administered cisplatin. In cases where a mammal having cancer is treated with one or more compounds that inhibit the function of an intracellular domain of PD-L1 and is treated with one or more cancer treatments, the one or more cancer treatments can be administered at the same time or independently. For example, the one or more compounds that inhibit the function of an intracellular domain of PD-L1 can be administered first, and the one or more cancer treatments administered second, or vice versa.

A compound that inhibits (e.g., reduces or eliminates) the function of an intracellular domain of PD-L1 can be any molecule that inhibits the binding between a PD-L1 intracellular domain and its target mRNA and/or HuR. A compound that inhibits the function of an intracellular domain of PD-L1 can be any appropriate type of molecule (e.g., nucleic acids such as siRNA molecules, shRNA molecules, antisense molecules, and miRNAs molecules, polypeptides (such as antibodies), and small molecules. In some cases, a compound that inhibits the function of an intracellular domain of PD-L1 can bind to the intracellular domain of PD-L1 to prevent binding between the PD-L1 intracellular domain and its target mRNA and/or HuR. For example, an antisense molecule, an antibody, and/or a small molecule can bind to the intracellular domain of PD-L1 (e.g., to prevent binding between the PD-L1 intracellular domain and its target mRNA and/or HuR). Examples of compounds that can bind to the intracellular domain of PD-L1 include, without limitation, molecules containing a PD-L1 binding RNA motif (e.g., GVAGAW where V is A, C, or G, and where W is A or U; SEQ ID NO:1). In some cases, a compound that inhibits the function of an intracellular domain of PD-L1 can lack the ability to bind to an extracellular domain of PD-L1.

In some cases, a compound that inhibits (e.g., reduces or eliminates) the function of an intracellular domain of PD-L1 can reduce or eliminate PD-L1 polypeptide expression. For example, nucleic acid molecules designed to induce RNA interference of PD-L1 (e.g., a siRNA molecule or a shRNA molecule) can eliminate or reduce PD-L1 polypeptide expression. Examples of compounds that can reduce or eliminate PD-L1 polypeptide expression include, without limitation, nucleic acid sequences encoding shRNA molecules targeting PD-L1. For example, an shRNA molecule targeting PD-L1 can include the sequence GACCUAUAUGUGGUAGAGUAU (SEQ ID NO:3). For example, an shRNA molecule targeting PD-L1 can include the sequence CGAAUUACUGUGAAAGUCAAU (SEQ ID NO:4).

In some cases, a compound that inhibits (e.g., reduces or eliminates) the function of an intracellular domain of PD-L1 can bind to the intracellular domain of PD-L1 to prevent binding between the PD-L1 intracellular domain and its target mRNA and/or HuR. For example, polypeptides designed to bind to the intracellular domain of PD-L1 to prevent binding between the PD-L1 intracellular domain and its target mRNA and/or HuR can eliminate or reduce binding between the PD-L1 intracellular domain and its target mRNA and/or HuR. Examples of compounds that can bind to the intracellular domain of PD-L1 to prevent binding between the PD-L1 intracellular domain and its target mRNA and/or HuR include, without limitation, polypeptide sequences that block the PD-L1 binding site for its target mRNA and/or HuR. A polypeptide sequence that can bind to the intracellular domain of PD-L1 to prevent binding between the PD-L1 intracellular domain and its target mRNA and/or HuR can include from about 9 amino acids to about 19 amino acids (e.g., from about 9 to about 17, from about 9 to about 15, from about 9 to about 12, from about 10 to about 19, from about 12 to about 19, from about 14 to about 19, from about 17 to about 19, from about 10 to about 18, from about 12 to about 15, from about 10 to about 12, from about 12 to about 15, or from about 15 to about 18 amino acids). For example, a polypeptide sequence that can bind to the intracellular domain of PD-L1 to prevent binding between the PD-L1 intracellular domain and its target mRNA and/or HuR can include about 10 amino acids. In some cases, a polypeptide sequence that can bind to the intracellular domain of PD-L1 to prevent binding between the PD-L1 intracellular domain and its target mRNA and/or HuR (e.g., NBS1 RNA) can have at least about 20% sequence identity (e.g., about 40% sequence identity, 50% sequence identity, 60% sequence identity, 70% sequence identity, 75% sequence identity, 80% sequence identity, 85% sequence identity, 90% sequence identity, 95% sequence identity, 97% sequence identity, 98% sequence identity, 99% sequence identity, or 100% sequence identity) to the sequence KKCGIQDTNS (SEQ ID NO:31). For example, a polypeptide sequence that can bind to the intracellular domain of PD-L1 to prevent binding between the PD-L1 intracellular domain and its target mRNA and/or HuR (e.g., NBS1 RNA) can include the sequence KKCGIQDTNS (SEQ ID NO:31).

In some cases, a compound that inhibits (e.g., reduces or eliminates) the function of an intracellular PD-L1 domain can include one or more additional features designed to enhance cellular uptake of the compound that inhibits the function of an intracellular PD-L1 domain. Examples of features that can enhance cellular uptake of the compound that inhibits the function of an intracellular PD-L1 domain include, without limitation, CPPs, targeting peptides, and nuclear localization signals. For example, a compound that inhibits the function of an intracellular PD-L1 domain can include one or more CPPs. Examples of CPPs that can be used as described herein include, without limitation, polypeptides having the amino acid sequence RRRRRRRR (SEQ ID NO:32), polypeptides having the amino acid sequence GRKKRRQRRRPQ (SEQ ID NO:33), polypeptides having the amino acid sequence RQIKIWFQNRRMKWKK (SEQ ID NO:34), and polypeptides having the amino acid sequence IAWVKAFIRKLRKGPLGGPLGIAGQRGDS (SEQ ID NO:35). In some cases, a CPP that can be used as described herein can be as described elsewhere (see, e.g., Kalafatovic et al., Molecules, 22:1929 (2017)). A feature designed to enhance cellular uptake of the compound that inhibits the function of an intracellular PD-L1 domain can be connected to the C-terminal end of the compound that inhibits the function of an intracellular PD-L1 domain, the N-terminal end of the compound that inhibits the function of an intracellular PD-L1 domain, or both the C-terminal end and the N-terminal end of the compound that inhibits (e.g., reduces or eliminates) the function of an intracellular PD-L1 domain.

A compound that inhibits (e.g., reduces or eliminates) the function of an intracellular PD-L1 domain can be identified as described herein. In some cases, methods for identifying a compound that inhibits the function of an intracellular domain of PD-L1 can include a cell-free assay. In some cases, methods for identifying a compound that inhibits the function of an intracellular domain of PD-L1 can include an assay using one or more cells expressing PD-L1 (e.g., endogenously expressing PD-L1 or recombinantly expression PD-L1). For example, a candidate compound can be contacted with at least part of a PD-L1 polypeptide (e.g., the intracellular domain), and the ability of the candidate compound to inhibit (e.g., reduce or eliminate) the function of an intracellular domain of PD-L1 (e.g., binding with its target mRNA and/or HuR) can be determined. The ability of a candidate compound to inhibit the function of an intracellular domain of PD-L1 can be determined using any appropriate method. For example, the binding of the intracellular domain of PD-L1 to its target mRNA (e.g., NBS1) and/or HuR in the presence and absence of a candidate compound can be determined using, for example, immunoprecipitation (e.g., co-immunoprecipitation), nuclear run-on assays, RNA immunoprecipitation, and/or gel-shift assays. In some cases, the ability of a candidate compound to inhibit the function of an intracellular domain of PD-L1 can be determined as described in, for example, Example 1.

The ability of a candidate compound to inhibit expression of PD-L1 can be determined using any appropriate method. For example, expression of PD-L1 in the presence and absence of a candidate compound can be determined using, for example, a RT-PCR (to evaluate transcription) and/or a western blot analysis (to evaluate translation). In some cases, the ability of a candidate compound to inhibit expression of PD-L1 can be determined as described in, for example, Example 1.

In some cases, one or more compounds that inhibit (e.g., reduce or eliminate) the function of an intracellular PD-L1 domain can be formulated into a pharmaceutically acceptable composition for administration to a mammal having cancer. For example, a therapeutically effective amount of one or more compounds that inhibit the function of an intracellular PD-L1 domain can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, nano-particles, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more compounds that inhibit the function of an intracellular PD-L1 domain can be designed for oral or parenteral (including subcutaneous, intramuscular, intravenous, and intradermal) administration. When being administered orally, a pharmaceutical composition containing one or more compounds that inhibit the function of an intracellular PD-L1 domain can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets. Such injection solutions can be in the form, for example, of a sterile injectable aqueous or oleaginous suspension. This suspension may be formulated using, for example, suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1, 3-butanediol. Examples of acceptable vehicles and solvents that can be used include, without limitation, mannitol, water, Ringer's solution, and isotonic sodium chloride solution. In addition, sterile, fixed oils can be used as a solvent or suspending medium. In some cases, a bland fixed oil can be used such as synthetic mono- or di-glycerides. Fatty acids, such as oleic acid and its glyceride derivatives can be used in the preparation of injectables, as can natural pharmaceutically-acceptable oils, such as olive oil or castor oil, including those in their polyoxyethylated versions. In some cases, these oil solutions or suspensions can contain a long-chain alcohol diluent or dispersant.

In some cases, a pharmaceutically acceptable composition including one or more compounds that inhibit the function of an intracellular PD-L1 domain can be administered locally or systemically. For example, a composition containing a compound that inhibits the function of an intracellular PD-L1 domain can be administered locally by injection into or near a cancer (e.g., a tumor) in a mammal (e.g., a human). For example, a composition containing a compound that inhibits the function of an intracellular PD-L1 domain can be administered systemically by oral administration or by injection (e.g., subcutaneous, intramuscular, intravenous, and intradermal injection) to a mammal (e.g., a human).

Effective doses can vary depending on the severity of the cancer, the route of administration, the age and general health condition of the subject, excipient usage, the possibility of co-usage with other therapeutic treatments such as use of other agents, and the judgment of the treating physician.

An effective amount of a composition containing one or more compounds that inhibit the function of an intracellular PD-L1 domain can be any amount that sensitizes cancer cells in the mammal to one or more cancer treatments (e.g., radiation therapy and/or chemotherapy) without producing significant toxicity to the mammal. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration can be any frequency that sensitizes cancer cells in the mammal to one or more cancer treatments (e.g., radiation therapy and/or chemotherapy) without producing significant toxicity to the mammal. For example, the frequency of administration can be from about once a week to about three times a day, or from about twice a month to about six times a day, or from about twice a week to about once a day. The frequency of administration can remain constant or can be variable during the duration of treatment. A course of treatment with a composition containing one or more compounds that inhibit the function of an intracellular PD-L1 domain can include rest periods. For example, a composition containing one or more compounds that inhibit the function of an intracellular PD-L1 domain can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing one or more compounds that inhibit the function of an intracellular PD-L1 domain can be any duration that sensitizes cancer cells in the mammal to one or more cancer treatments (e.g., radiation therapy and/or chemotherapy) without producing significant toxicity to the mammal. Thus, the effective duration can vary from several days to several weeks, months, or years. In general, the effective duration for the treatment of cancer can range in duration from six months to one year. Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment and the severity of one or more symptoms related to the condition being treated (e.g., cancer) can be monitored. Any appropriate method can be used to determine whether or not the sensitivity of cancer cells in the mammal to one or more cancer treatments (e.g., radiation therapy and/or chemotherapy) is increased. For example, the responsiveness of cancer (e.g., based on the size and/or number of tumors) can be assessed using office imaging techniques at different time points.

The invention will be further described in the following examples, which do not limit the scope of the invention described in the claims.

### EXAMPLES

### Example 1: PD-L1 (B7-H1) Regulates the DNA Damage Response and mRNA Stability

### Materials and Methods

### Cell Lines, PD-L1 Knockout Mice, Antibodies and Plasmid

MDA-MB-231 cells were cultured in Dulbecco's modified Eagle's medium (DMEM) with 10% Fetal Bovine Serum (FBS). HCT116 cells were cultured in McCoy's 5 A with 10% FBS.

PD-L1 knockout mice, originally created from BALB/C mice, were obtained from Dr. Haidong Dong' lab.

GAPDH antibody was purchased from Sigma-Aldrich. PD-L1 antibody was purchased from cell signaling Technology (CST). HuR antibody was purchased from Abcam. NBS1 antibody was purchased from Bethyl Lab.

shRNAs that target PD-L1 and HuR were purchased from Mayo Clinic RNA interference shared resource (RISR), and were inserted into pLKO.1-puro vectors. The sequence information of shRNAs for PD-L1 and for HuR is as follows.

| | | SEQ |
|---|---|---|
| | DNA sequence | ID |
| | encoding the shRNA | NO: |
| PD-L1 | | |
| shRNA-1 | GACCTATATGTGGTAGAGTAT | 5 |
| shRNA-2 | CGAATTACTGTGAAAGTCAAT | 6 |
| HuR | | |
| shRNA-1 | CGTGGATCAGACTACAGGTTT | 7 |
| shRNA-2 | ACCATGACAAACTATGAAGAA | 8 |

The NBS1 RNA sequence was cloned into pGEMT-easy (Promega) vector, which was used for in vitro transcription. The RNA motif for dual-fluorescent reporter was cloned into pmirGLO (Promega). The full length of truncations of PD-L1 and HuR were cloned into plvx3-puro and pCMV-HA vectors, respectively.

### Dual Reporter Luciferase Assay

Dual reporter luciferase assay was performed using Dual-Luciferase® Reporter Assay System according to manufacturer's instructions (Promega).

### RNA Extraction, Reverse Transcription and Quantitative RT-PCR

RNA extraction was performed using quick-RNA™ miniPrep kit (zymo) according to the manufacturer's instructions. Reverse transcription was performed with PrimeScript RT-PCR Kit (Takara). Quantitative RT-PCR was performed using SYBR Green PCR Master Mix (Applied Biosystems).

Primers Used for qRT-PCR

| | | SEQ |
|---|---|---|
| | | ID |
| Primer | Sequence | NO: |
| NBS1 exon 1-2 forward | GCAGGAGGAGAACCATACAGAC | 9 |
| NBS1 exon 1-2 reverse | ATGATTTCGGCTGATCGACT | 10 |
| NBS1 exon 2-3 forward | CCAACCTGAGTCAAACAGATGA | 11 |
| NBS1 exon 2-3 reverse | AAGTAATACCATCCCCCGACTT | 12 |
| NBS1 exon 5-7 forward | TGAAAGCAGTTGAGTCCAAGAA | 13 |
| NBS1 exon 5-7 reverse | AAAGACAACTGCGGAACTCAAT | 14 |
| NBS1 exon 11-12 forward | CAATGTTAGAAAAAGGCCAAGG | 15 |
| NBS1 exon 11-12 reverse | GTTCACGTTTCTTCCCAATTTC | 16 |
| NBS1 exon 8-10 forward | GCGGTGATTTTCATGACTACAA | 17 |
| NBS1 exon 8-10 reverse | AAATCCCATGTATCTGCTTGCT | 18 |
| NBS1 exon 16 forward | TGCAGTGTTCTACACCTTGCTT | 19 |
| NBS1 exon 16 reverse | ACTGAAGCCATTTTGTTTGGAT | 20 |
| GAPDH forward | CGGAGTCAACGGATTTGGTCGT | 21 |
| GAPDH reverse | TCTCAGCCTTGACGGTGCCA | 22 |
| BRCA1 forward | ATGGAAGGTAAAGAACCTGCAA | 23 |
| BRCA1 reverse | TGGAAGGCTAGGATTGACAAAT | 24 |
| POLQ forward | GACCTGCAAAGAGCAATGAAG | 25 |
| POLQ reverse | ACCCCTCTTCAACTCCCACTA | 26 |
| FANCL forward | GTGGTAAAACCCCTGGGAAT | 27 |
| FANCL reverse | AGGATAGCACGAGCTGGAAA | 28 |
| ATM forward | TTTGCTTGAGGCTGATCCTT | 29 |
| ATM reverse | GATTGACTCTGCAGCCAACA | 30 |

### RNA Immunoprecipitation and RNA Pull Down

Native RNA immunoprecipitation (RIP) was performed using Magna RIP™ RNA-Binding Protein Immunoprecipitation Kit (Millipore) according to the manufacturer's instructions. Crosslinked RIP was performed as described elsewhere (see, e.g., Gilbert et al., RNA immunoprecipitation for determining RNA-protein associations in vivo. Curr Protoc Mol Biol Chapter 27, Unit 27 (2006)) with minor modification. Briefly, cells were first fixed with 0.3% formaldehyde for 10 minutes followed by stopping crosslinking reaction with 0.2M glycine. Then, the cells were lysed with FA lysis buffer (50 mM Tris HCl, pH=7.5, 140 mM NaCl, 1 mM EDTA, 1% (v/v) Triton X-100, 0.1% (w/v) sodium deoxycholate) followed by sonication (10% AMP, 5 second sonication, 5 second pause, 10 cycles). The supernatant of the cell lysate was then mixed with antibody and protein A/G magnetic beads followed by incubation overnight at 4° C. Then, the beads were washed two times of FA lysis buffer, FA500 buffer (50 mM Tris HCl, pH=7.5, 500 mM NaCl, 1 mM EDTA, 1% (v/v) Triton X-100, 0.1% (w/v) sodium deoxycholate), LiCl buffer (10 mM Tris HCl, pH 8, 250 mM LiCl, 0.5% (v/v) NP-40, 0.1% (w/v) sodium deoxycholate, 1 mM EDTA), and TE/100 mM NaCl buffer (10 mM Tris HCl, pH 8, 1 mM EDTA, 100 mM NaCl). The RNA was then eluted by elution buffer (100 mM Tris-Cl, pH 8, 10 mM EDTA, 1% (w/v) SDS) and extracted using TRIzol™ (Invitrogen).

RNA pull down was performed as described elsewhere (see, e.g., Tsai et al., Science 329:689-693 (2010)) with minor modification. In short, in vitro biotin labeled RNAs were mixed with MDA-MB-231 lysate, followed by targeting RNAs-protein complex with streptavidin beads. The co-precipitated proteins were then performed western blot for analysis.

### Immunofluorescence

The immunofluorescence was performed as described elsewhere (see, e.g., Huang et al., Science 314: 294-297 (2006)) with minor modification. Briefly, cells were first fixed by 3% paraformaldehyde followed by permeabilization with 0.5% Triton X-100, then cells were blocked by 5% goat serum followed by incubating with primary antibody. Fluorescent secondary antibody and DAPI were then incubated with cells to stain the targeted proteins and nucleus. The data was analyzed by fluorescent microscopy.

### Co-Immunoprecipitation

Cells were first lysed by NETN buffer (20 mM Tris-HCl, pH 8.0, 150 mM NaCl, 1 mM, EDTA, 0.5% Nonidet P-40). 3 μg antibody and protein A/G beads then were added into cell lysate. After overnight of incubation, the beads were washed 3-5 times with NETN. The co-immunoprecipitated protein was then analyzed by western blot.

### Nuclear Run on

Nuclear run on was performed as described elsewhere (see, e.g., Patrone et al., Biotechniques 29:1012-1014 (2000)) with minor modification. Briefly, cell nuclei were extracted using nuclear extraction buffer (10 mM Tris-HCl, pH 7.4, 3 mM, MgCl2, 10 mM NaCl, 150 mM sucrose and 0.5% NP-40) followed by suspended in freezing buffer (50 mM Tris-HCl, pH 8.3, 40% glycerol, 5 mM MgCl2 and 0.1 mM EDTA), then the nuclei were mixed with same volume of 2× transcription buffer (200 mM KCl, 20 mM Tris-HCl, pH 8.0, 5 mM MgCl₂, 4 mM dithiothreitol (DTT), 4 mM each of ATP, GTP and CTP, 1 mM biotin-16-UTP, 200 mM sucrose and 20% glycerol). After a 30-minute incubation at 30° C., the de novo synthesized transcripts were purified by Dynabeads M-280 (Invitrogen). The purified RNAs were then performed reverse transcription and quantitation.

### RIP-Seq and RNA-Seq

RIP seq was performed using an Illumina HiSeq 2000 sequencer at the Genomic Core facility of the Mayo Clinic, Rochester, Minn. Paired-end sequencing libraries were prepared using the TruSeq Stranded Total Sample Preparation kit (Illumina) by the Mayo Clinic sequencing core facilities followed by quality control, cluster generation, and sequencing on the Illumina HiSeq 2000 platform. Sequence alignment was performed using TopHat 2.0.14 (Kim et al., Genome Biol 14:R36 (2013)) against the hg19 human reference genome. Then, Peak calling and annotation were performed using HOMER 4.8.3 (Heinz et al., Mol Cell 38: 576-589 (2010)). The PD-L1 binding RNAs were defined by at least 5 folds higher in PD-L1 group compared to IgG. Motif scan was performed on peaks called by HOMER for each sample using MEME-ChIP (Machanick et al., Bioinformatics 27:1696-1697 (2011)) against the HOCOMOCO transcription factor binding site database (Kulakovskiy et al., Nucl Acids Res 41:D195-D202 (2013)).

RNA seq was performed using an Illumina HiSeq 2000 sequencer. Paired-end sequencing libraries were prepared using the TruSeq Stranded Total Sample Preparation kit (Illumina) followed by quality control, cluster generation, and sequencing on the Illumina HiSeq 2000 platform. RNA-Seq results were delivered as BAM files, which were converted to FASTQ format using Picard. FASTQ files were aligned to the hg19 human reference genome using TopHat 2.0.14 (Kim et al., Genome Biol 14:R36 (2013)). Expression values were calculated with featureCounts v1.4.6-p 2 (Liao et al., Bioinformatics 30:923-930 (2014)), and differential expression analysis was determined by DESeq2 (Love et al., Genome Biol 15:550 (2014)). The downregulated genes for RNA seq were defined by counts>100, folds>1.5 and p<0.02.

Go analysis was performed using Gene Ontology Consortium website (http://www.geneontology.org/).

### Results

The binding of PD-L1 to its receptor, PD-1 transmits signals that inhibit T-cell activation. Therefore, abrogating the PD-1/PD-L1 interaction in multiple malignancies has emerged as an effective therapeutic strategy to enhance antitumor immunity. There is considerable interest in combining DNA damaging chemotherapy or radiotherapy with drugs that target PD-L1 with the goal of creating a more immunogenic tumor microenvironment, thereby enhancing the anti-tumor immune response (Kang et al., J Immunother Cancer 4:51 (2016); Daly et al., J Thorac Oncol 10:1685-1693 (2015); and Sharma et al., Science 348: 56-61 (2015)).

### PD-L1 and DNA Damage

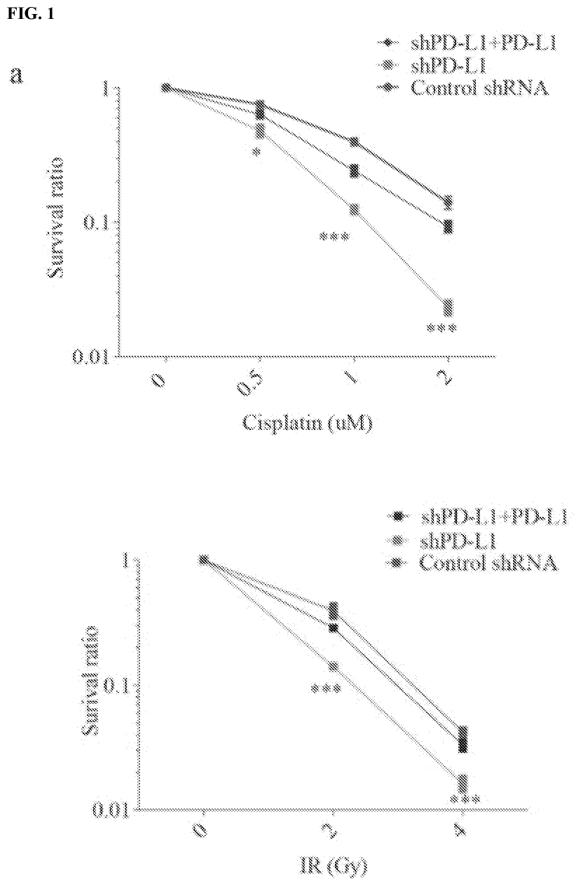

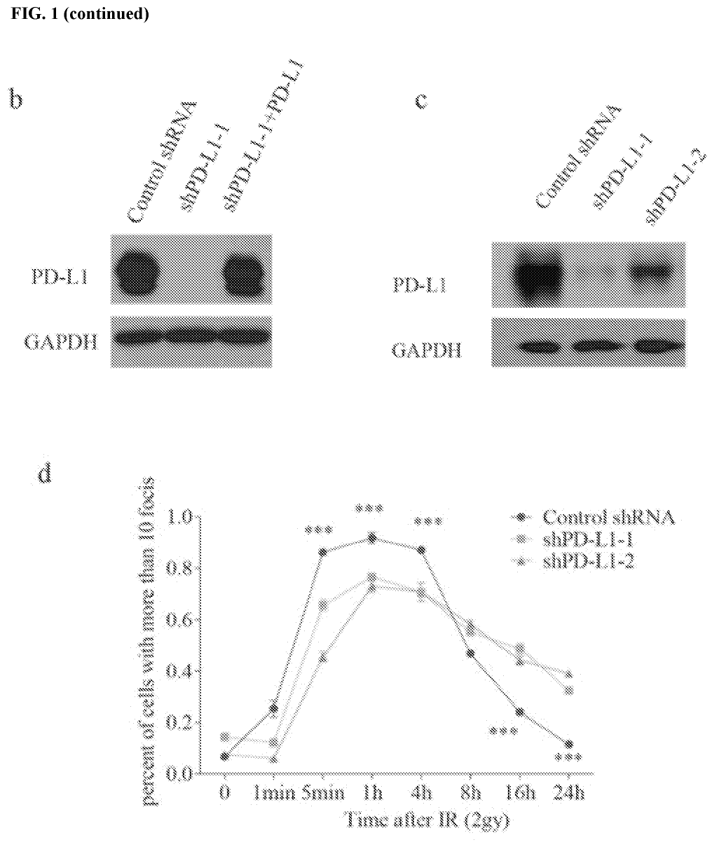

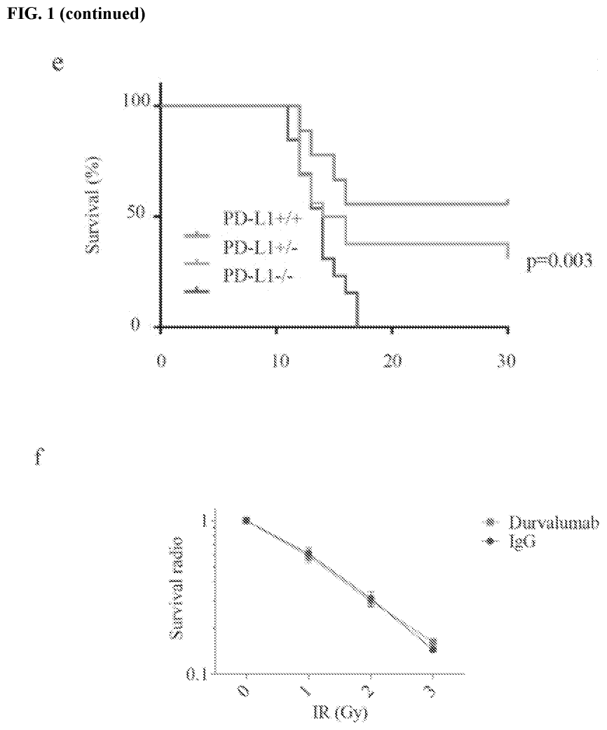

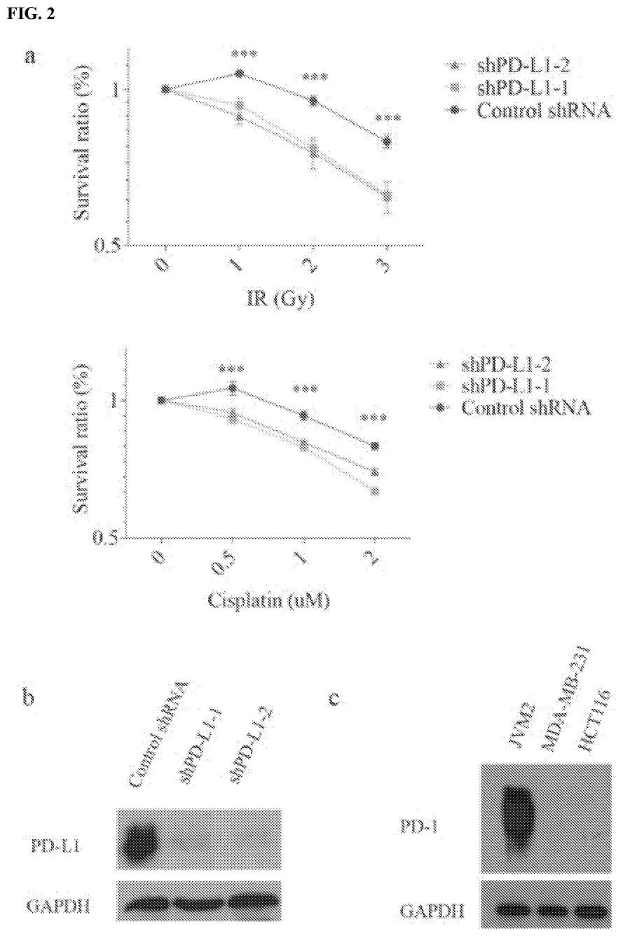

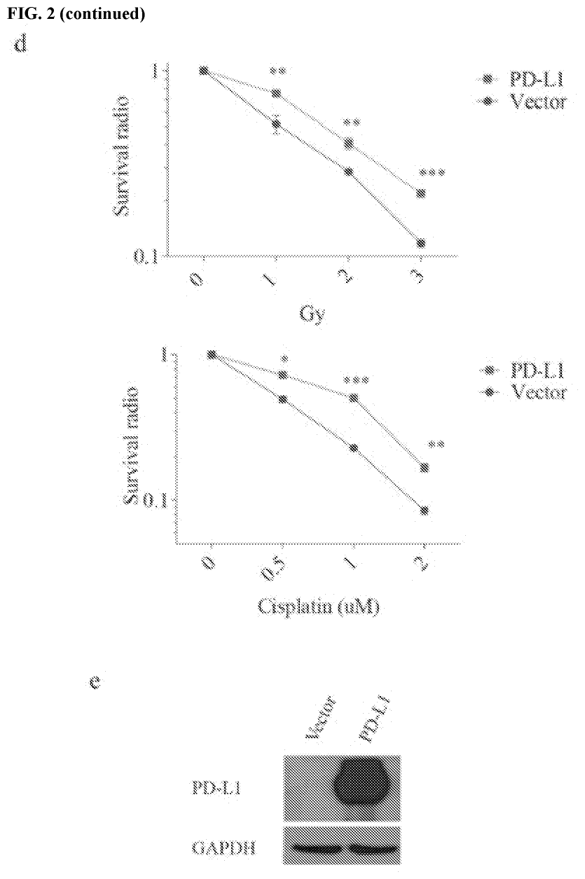

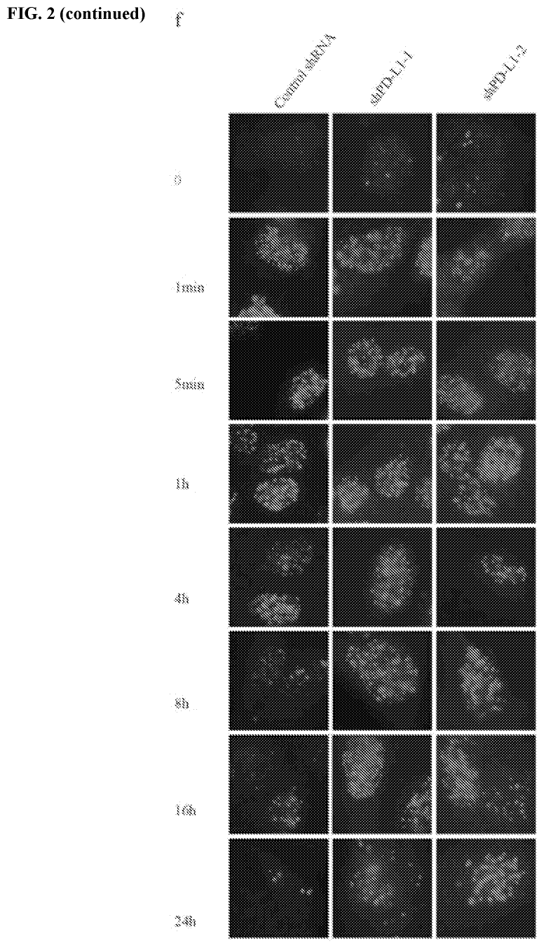

As demonstrated herein, it was observed that PD-L1 depletion with two independent shRNAs sensitized HCT116 and MDA-MB-231 cells to cisplatin and ionizing radiation (IR) in vitro, in the absence of immune effects (FIG. 1a-b and FIG. 2a-b). Meanwhile, restoration of PD-L1 expression in knockdown cells reversed this phenotype (FIG. 1a-b), suggesting that the observed impact on platinum and radiosensitivity by shRNAs was specific. The expression of PD-1 in HCT116 and MDA-MB-231 was not detectable (FIG. 2c), suggesting that PD-L1 might have a cell intrinsic function in the response to chemotherapy and radiation. Meanwhile, the effect was also examined in HeLa cells, which have very low expression of PD-L1. Exogenously expressing PD-L1 in HeLa cells increased their resistance to IR and cisplatin (FIG. 2d-e), further suggesting the function of PD-L1 in DNA damage response.

Cisplatin and radiation kill cancer cells by inducing DNA damage. Therefore, it was evaluated whether DNA repair function is compromised in cells depleted of PD-L1. Notably, through immunofluorescence, it was found that the formation of rH2AX foci was more sustained after PD-L1 depletion (FIG. 1c-d, FIG. 2f), indicative of compromised DNA repair function in PD-L1 knockdown cells.

Next, the impact of PD-L1 on sensitivity to DNA damage in vivo was assessed by using PD-L1 knockout mice. PD-L1 knockout BALB/C mice demonstrated profound radiosensitivity, with significantly decreased survival following whole body irradiation compared to the wild-type control (FIG. 1e, p=0.003).

Taken together, these results provided strong evidence of a link between PD-L1 and the cellular response to DNA damage. Of note, the clinically available PD-L1 monoclonal antibody durvalumab did not sensitize cells to IR, suggesting that PD-L1 function in regulating the DDR might occur intracellularly (FIG. 1f).

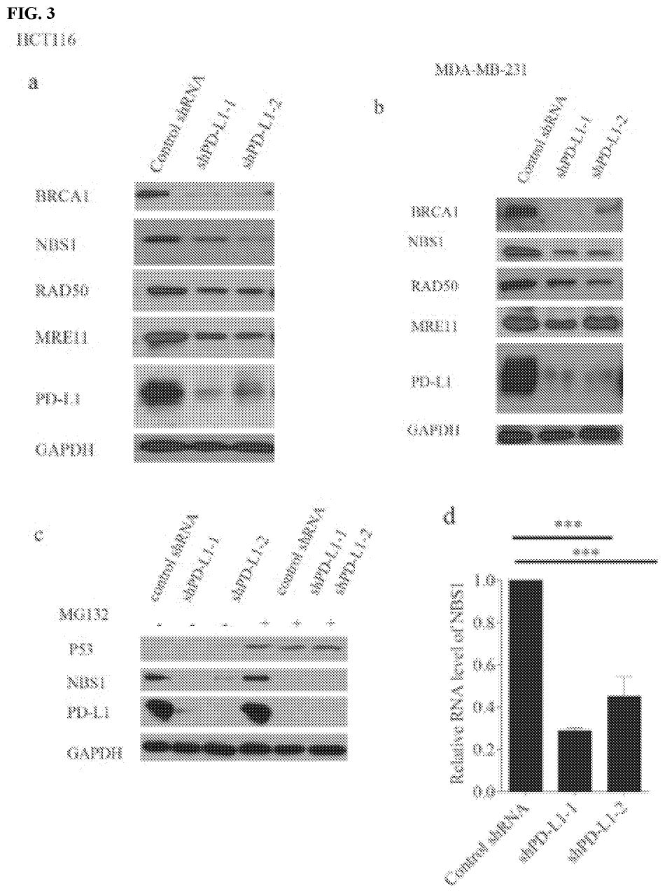

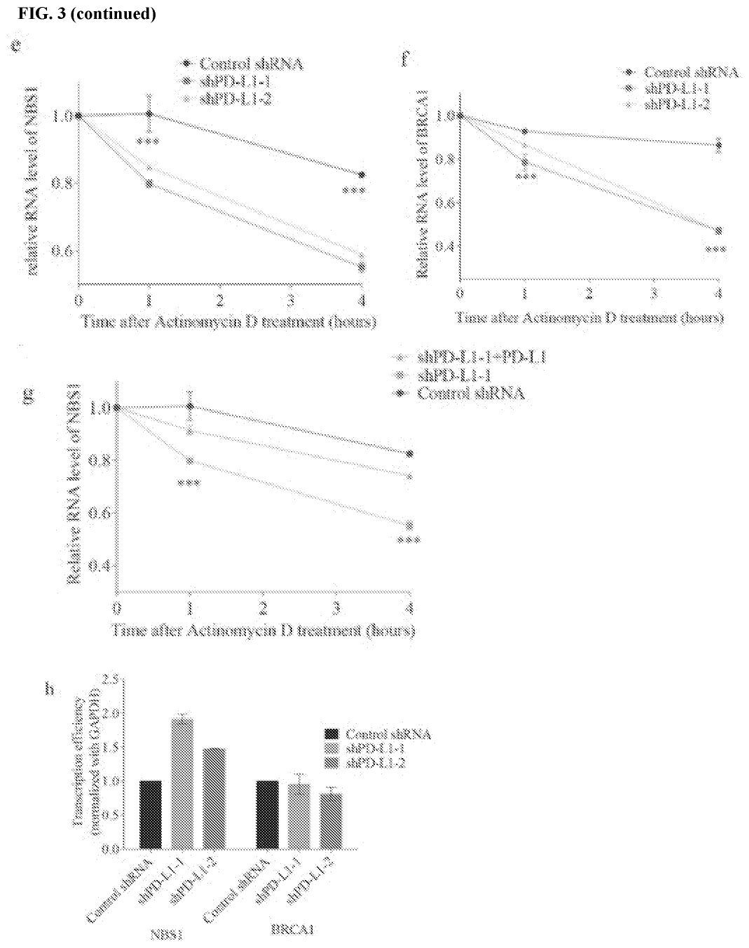

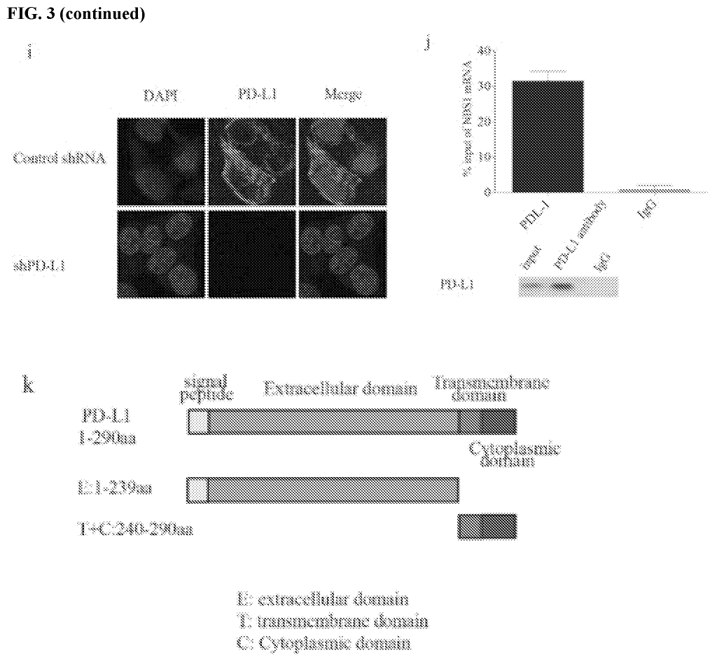

In order to mechanistically determine how PD-L1 affects the cellular response to DNA damage, whether PD-L1 depletion impacted the expression of any important genes in the DDR pathway in tumor cells was assessed. Notably, it was found that protein levels of all members of Mre11-Rad5O-Nbs1 (MRN) complex and BRCA1 decreased following knockdown of PD-L1 in both HCT116 and MDA-MB-231 cells (FIGS. 3a and b). NBS1 expression and BRCA1 expression were further examined to understand how PD-L1 regulates gene expression in subsequent studies.

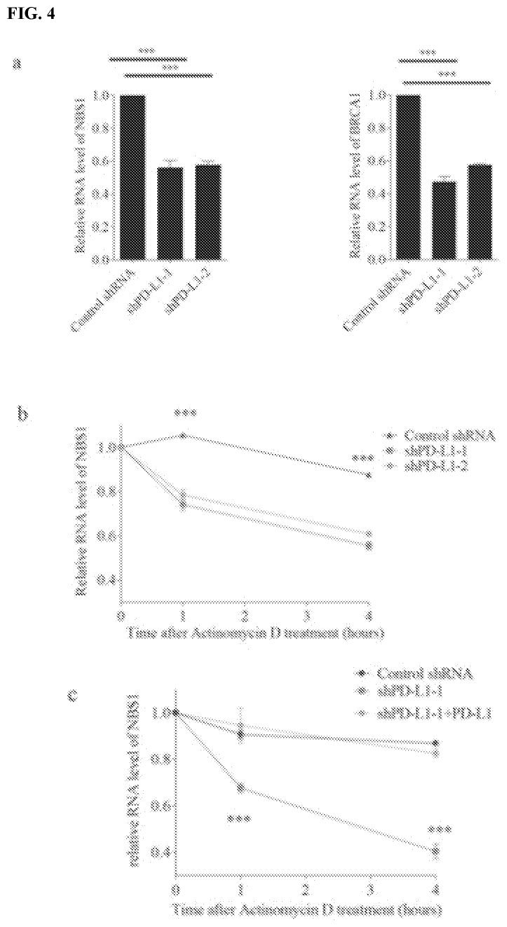

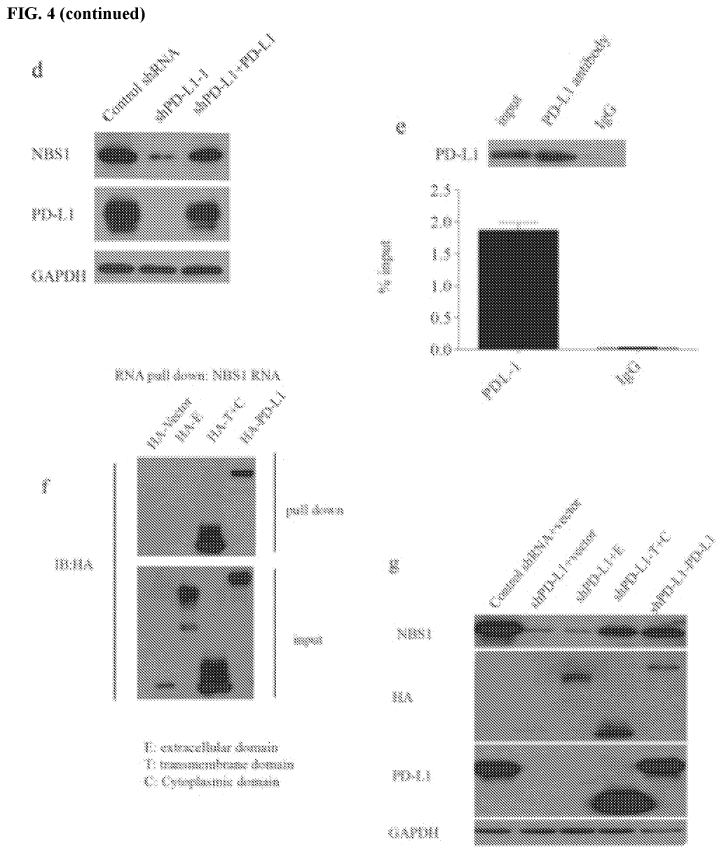

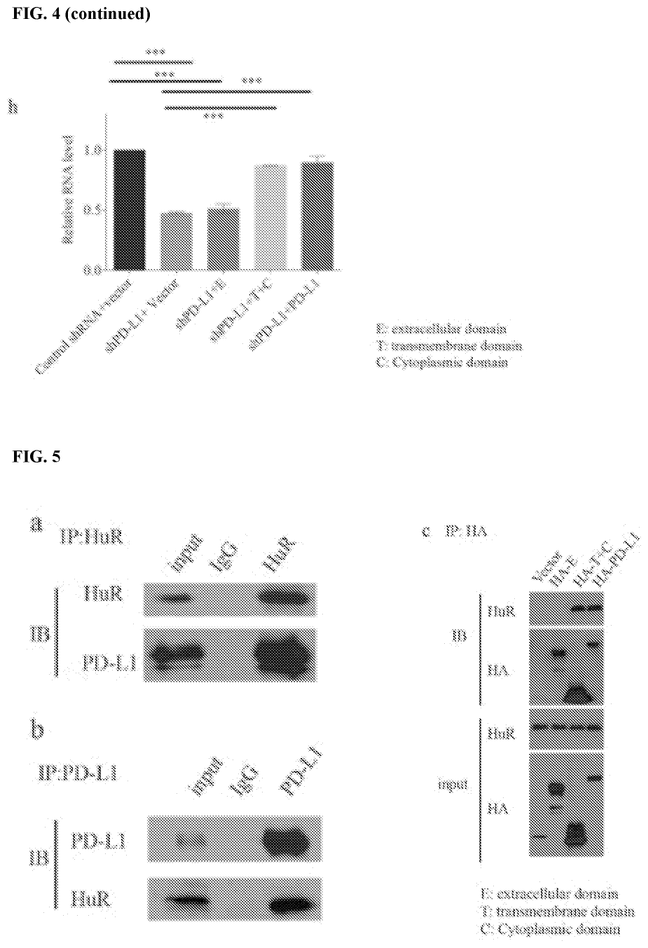

First, control and PD-L1 depleted MDA-MB-231 cells were treated with the proteasome inhibitor MG132. The level of NBS1 did not recover after MG132 treatment, implying the reduced NBS1 level observed in PD-L1 depleted cells did not result from increased protein degradation through the proteasome (FIG. 3c). However, it was observed that the mRNA level of NBS1 was significantly decreased (FIG. 4a, FIG. 3d). Moreover, BRCA1 mRNA levels were similarly reduced after PD-L1 knockdown (FIG. 4a).

PD-L1 and mRNA Stability

In eukaryotic cells, mRNA homeostasis is achieved through a balance between mRNA synthesis and degradation. Therefore, whether PD-L1 impacted NBS1 and BRCA1 mRNA stability or transcription was investigated. Control and PD-L1 depleted MDA-MB-231 and HCT116 cells were treated with the transcriptional inhibitor actinomycin D, and NBS1 and BRCA1 mRNA levels were assessed over time. The half-life of both NBS1 and BRCA1 mRNA was significantly shorter in PD-L1 depleted cells compared with control (FIG. 4b, FIGS. 3e and f), and restoration of PD-L1 expression restored NBS1 mRNA stability (FIG. 4c-d, FIG. 3g). A nuclear run-on assay was used to assess whether reduced NBS1 and BRCA1 mRNA in PD-L1 depleted cells were due to decreased gene transcription. However, the transcription of NBS1 and BRCA1 was either unchanged or increased in PD-L1 knockdown conditions (FIG. 3h), suggesting that decreased NBS1 and BRCA1 mRNA levels in PD-L1-deficient cells is due to reduced mRNA stability, rather than transcription.

To define how PD-L1 regulates mRNA stability, the subcellular localization of PD-L1 was confirmed using immunofluorescence. Notably, strong staining of PD-L1 was exhibited in the cytoplasm of HCT116 cells, and nuclear PD-L1 was also detectable (FIG. 3i). Next, using RNA immunoprecipitation (RIP), it was found that PD-L1 could interact with NBS1 mRNA under both native and crosslinking conditions (FIG. 4e and FIG. 3j). PD-L1 contains three domains (FIG. 3k): extracellular domain, transmembrane domain, and cytoplasmic domain. The extracellular domain is the largest domain of the protein and has already been solved by crystal analysis (Lin et al. Proc Natl Acad Sci USA 105: 3011-3016 (2008)). The transmembrane domain and cytoplasmic domain are both very small, and to date, their structures have not yet to be resolved. In order to identify which domain was responsible for binding with mRNA, two PD-L1 truncations were constructed: a first containing the extracellular domain (labeled E), and a second containing the transmembrane domain plus cytoplasmic domain (labeled T+C) (FIG. 3k). An RNA pull-down assay was performed using in vitro biotin labeled NBS1 RNAs. It was observed that the transmembrane and cytoplasmic domains of PD-L1 had strong binding affinity with NBS1 mRNA, while the extracellular domain of PD-L1 alone did not bind with NBS1 mRNA (FIG. 40. In addition, re-expression of the PD-L1 transmembrane and cytoplasmic domains rescued both NBS1 protein and mRNA levels in PD-L1-depleted cells, similar to the re-expression of full length PD-L1. However, re-expression of the PD-L1 extracellular domain alone had no effect (FIG. 4g-h). All of the above data suggest that PD-L1 binds and regulates NBS1 mRNA stability and the transmembrane and cytoplasmic domains of PD-L1 are required for binding with RNAs.

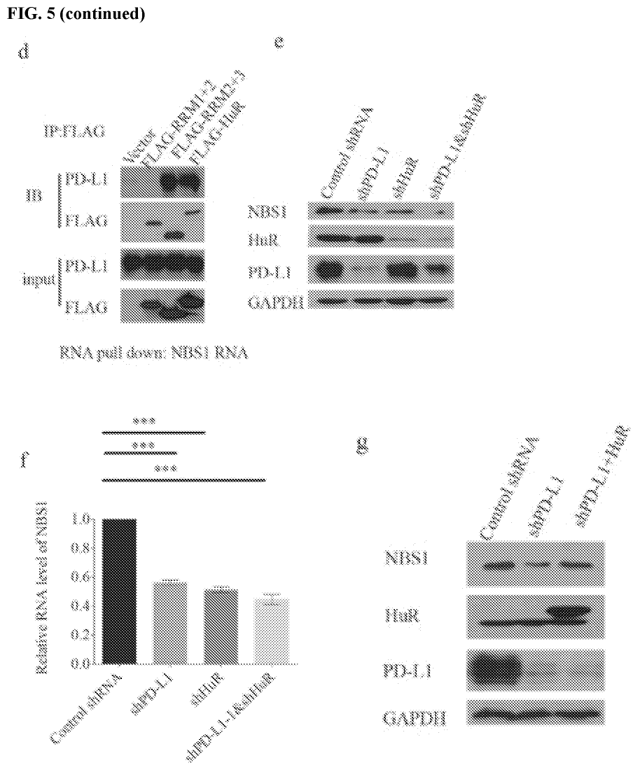

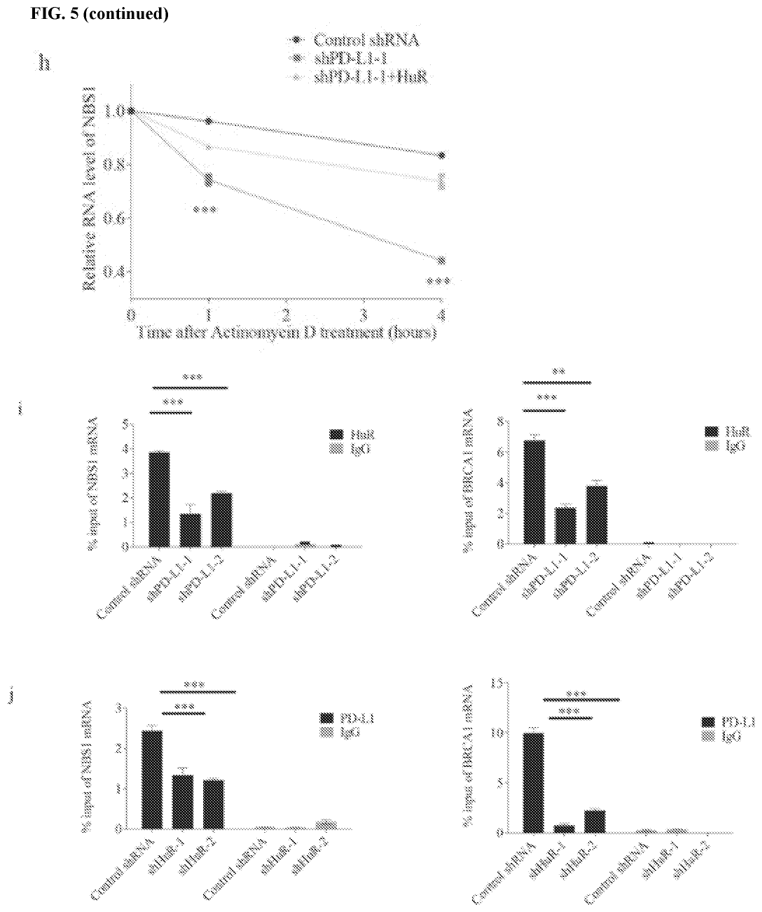

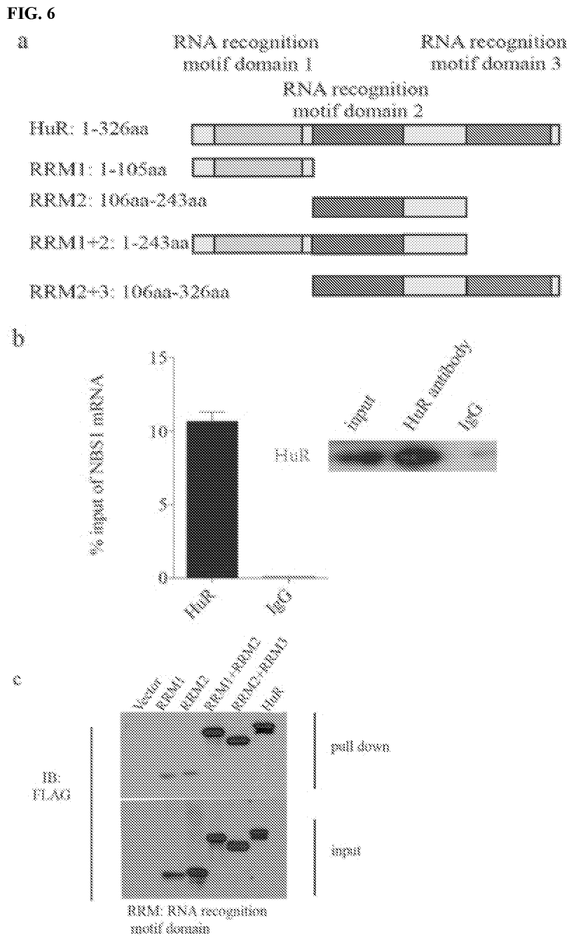

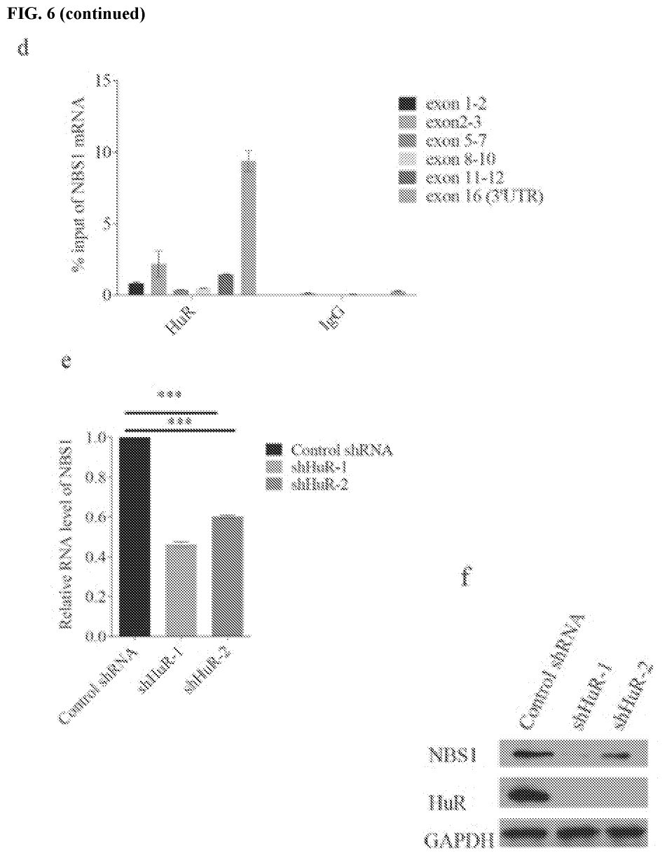

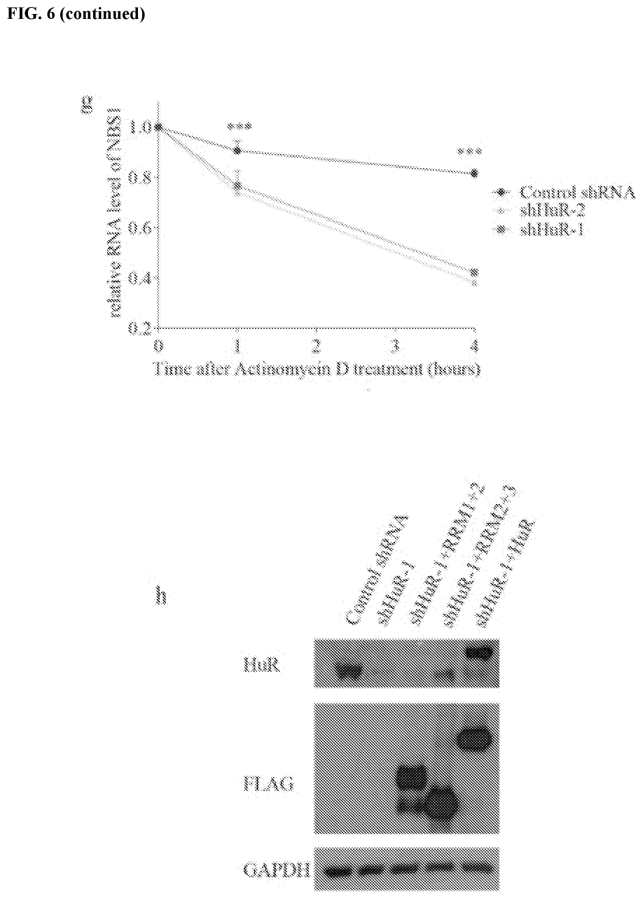

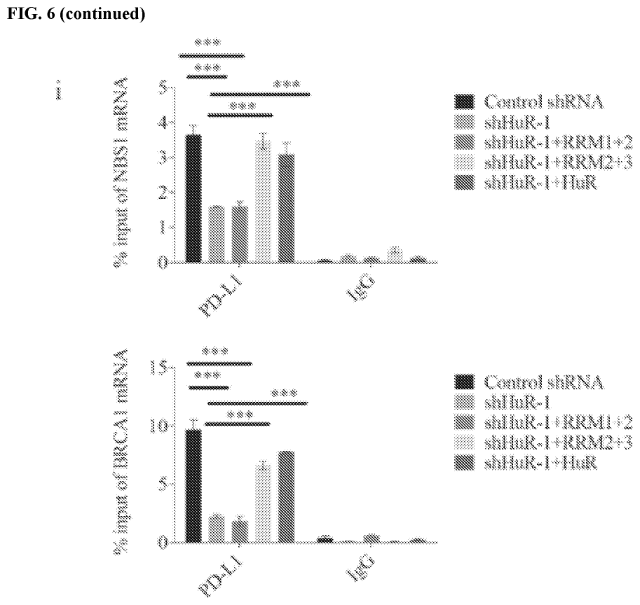

The stability of RNAs are regulated by many RNA binding proteins, which in turn affect the functions of one or several RNA-degrading enzymes like ribonucleases or RNases (Houseley et al., Cell 136:763-776 (2009)). To assess whether PD-L1 regulates mRNA stability through known RNA binding proteins, several different RNA binding proteins that have been previously shown to regulate RNA stability were tested. It was found that one RNA regulator, human antigen R (HuR), interacts with PD-L1 using co-immunoprecipitation (co-IP) assay (FIG. 5a-b). The co-IP was performed under the treatment of RNase A (250 μg/mL), suggesting the binding is not RNA-dependent. HuR is well known as a RNA binding protein, which has a preference to bind AU-rich region of targeted RNAs and regulates multiple RNAs' stability or translation (Wang et al., Int J Mol Sci 14:10015-10041 (2013)). HuR has three RNA recognition motif domains (RRM), with linker regions connecting each other (FIG. 6a; and Fan et al., Proc Natl Acad Sci USA 95:15293-15298 (1998)). In order to identify the binding site between PD-L1 and HuR, truncations of HuR (labeled RRM1, RRM2, RRM1+2 and RRM2+3) were constructed (FIG. 6a). Through co-IP assay, it was found that PD-L1 binds with HuR through its transmembrane and cytoplasmic domain (T+C) (FIG. 5c). RRM2+3, but not RMM1+2, was able to interact with PD-L1, suggesting that HuR binds with PD-L1 through its RRM3 domain (FIG. 5d).

To determine if PD-L1 and HuR might work collaboratively to regulate mRNA stability, RIP and RNA pull down assays were performed, both of which showed that HuR could interact with NBS1 mRNA (FIG. 6b-c). Furthermore, using the previously described HuR truncation mutants, it was found that all HuR RRM domains (1, 2, and 3) could interact with NBS1 RNA, while the 3′UTR of NBS1 mRNA was the primary HuR binding region (FIGS. 6c and d). Like the phenotype observed when cells were depleted of PD-L1, it was also found that depletion of HuR significantly decreased both protein and RNA level of NBS1, and destabilized NBS1 mRNA (FIG. 6e-g). Interestingly, simultaneous knockdown of both PD-L1 and HuR exhibited a similar effect as knockdown of either protein alone, decreasing NBS1 mRNA and protein level (FIGS. 5e and f). Overexpression of HuR partially reversed the decrease of NBS1 mRNA stability and protein level caused by PD-L1 depletion (FIGS. 5g and h). These results suggest that PD-L1 and HuR function in a common pathway to regulate mRNA stability.

To further investigate how PD-L1 and HuR regulate mRNA stability, the binding affinity of HuR with NBS1 and BRCA1 mRNA under PD-L1 depleted conditions was examined. Knockdown of PD-L1 significantly decreased the binding affinity of HuR with NBS1 and BRCA1 mRNA (FIG. 5i). Meanwhile, depletion of HuR also diminished the binding affinity of PD-L1 with NBS1 and BRCA1 mRNA (FIG. 5j), suggesting that PD-L1 and HuR strengthen each other's binding to mRNA. In MDA-MB-231 cells with complete HuR depletion, RRM1+2, RRM2+3, or full length HuR was ectopically re-expressed. RIP assay was performed to confirm the function domain of HuR. The result suggested that RRM2+3 and full length HuR can rescue the decreased binding affinity of PD-L1 with NBS1 and BRCA1 mRNA, but not the RRM1+2, which did not interact with PD-L1 (FIG. 6h-i). These results suggest that binding between HuR and PD-L1 is important for their regulation of gene expression. Together, these results suggest that PD-L1 and HuR collaborate to perform their function to regulate RNA stability.

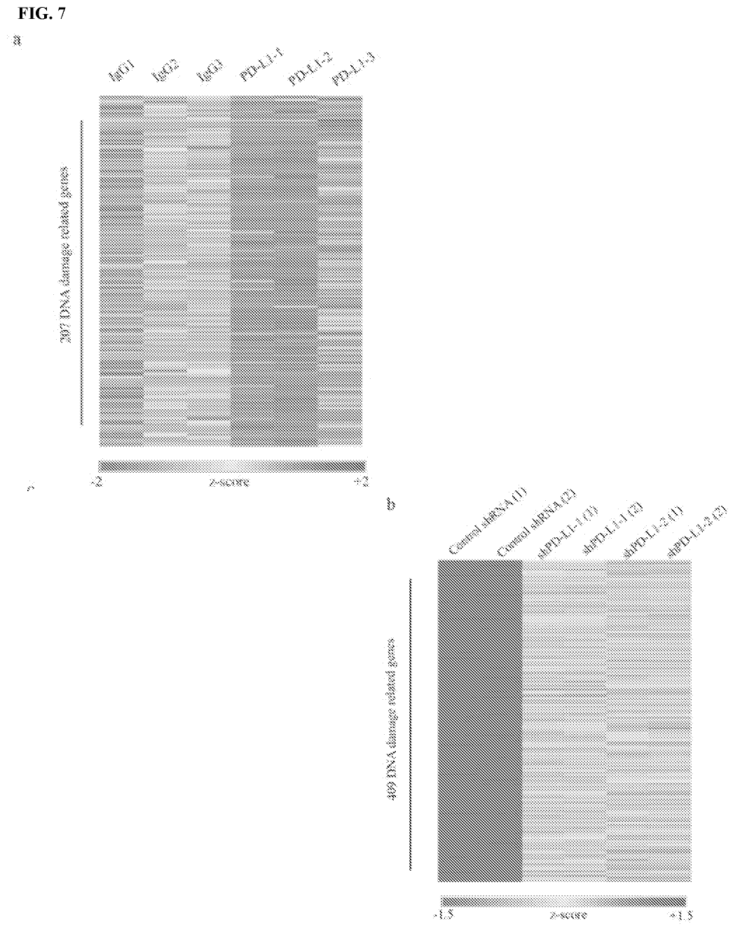

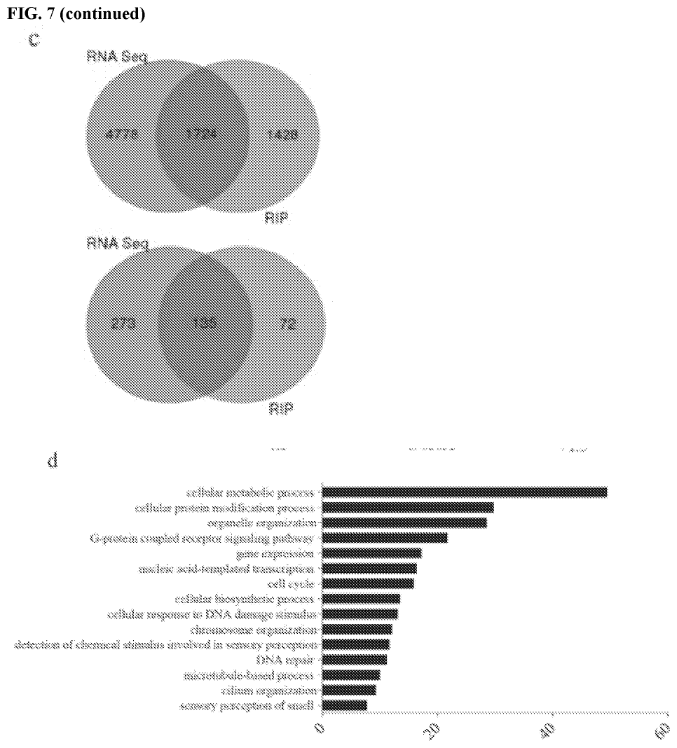

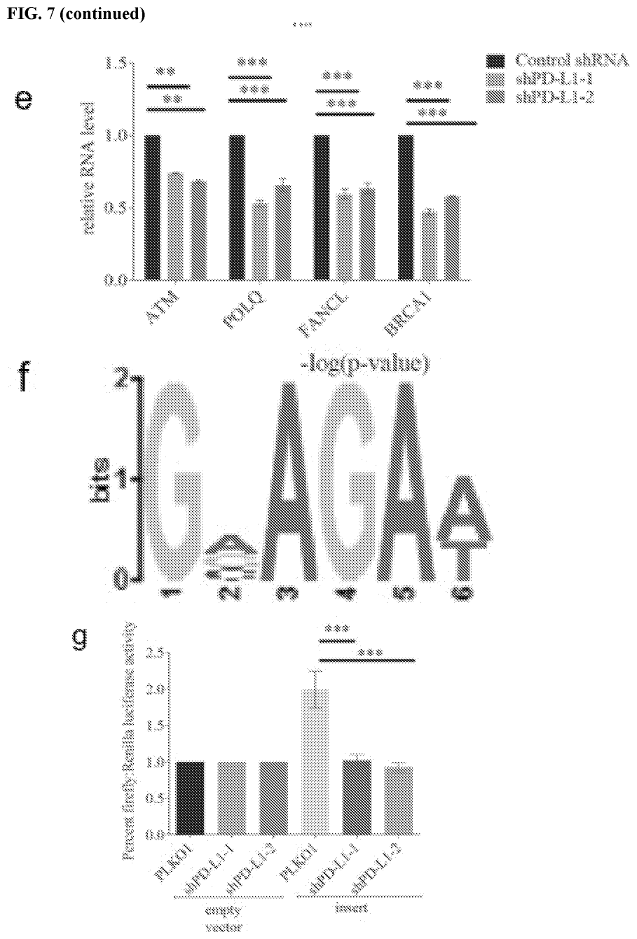

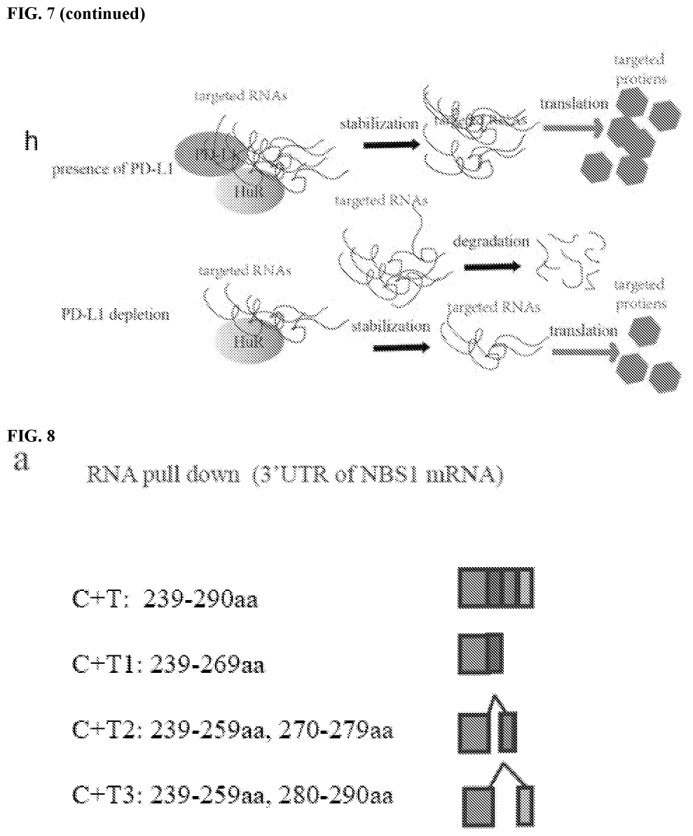

The above results suggest that PD-L1 binds mRNA and forms a complex with HuR, collaborating together to stabilize target mRNAs. To examine whether PD-L1 may regulate mRNA stability at genome-wide, cross-linked RIP-seq was performed using PD-L1 antibody to identify RNA transcripts that interact with PD-L1. A total of 3152 candidate RNAs were significantly enriched by PD-L1 compared to IgG, including NBS1 and BRCA1 mRNA (FIG. 7c, Tables 1-2). In order to assess the impact of these interactions on the broader transcriptome, PD-L1 was depleted, and RNA sequencing (RNA-seq) was performed. 6502 genes were identified that were downregulated under PD-L1 knockdown condition compared with control (counts>100, fold>1.5 and p-value<0.02), among which 1724 genes were identified for both datasets. Through Gene Ontology (GO) pathway analysis, it was confirmed that 408 genes from RNA-seq and 207 genes from RIP-seq were enriched in the ‘cellular response to DNA damage stimulus’ pathway (FIGS. 7a and b), respectively, among which 135 genes were enriched in both datasets (FIG. 7c). In addition to DNA damage pathway, many other pathways such as cell metabolism, transcription, cell cycle, and protein modification were significantly enriched in this assay, suggesting PD-L1 may regulate RNAs or gene expressions in multiple cellular processes (FIG. 7d). In order to confirm the accuracy of the sequencing data, the mRNA level of several DNA damage related genes was measured using quantitative reverse transcription PCR (qRT-PCR). The results showed that ATM, POLQ, FANCL, and BRCA1 mRNA levels were decreased under PD-L1 knockdown, suggesting the sequencing data is valid (FIG. 7e).

In order to determine the RNA sequence preference of PD-L1 in binding to RNA, MEME-ChIP (Machanick et al., Bioinformatics 27:1696-1697 (2011)) was performed to analyze the RIP-seq data. These results showed that sequence ‘GVAGAW’ (where V is A, C, or G, and where W is A or U; SEQ ID NO:1) was on the top of the list (FIG. 7f). To validate that this sequence is recognized by PD-L1, two copies of the sequence (GAAGAAGAAGAT; SEQ ID NO:2) were inserted into the dual-luciferase reporter vector, pmirGLO, and dual luciferase reporter assay was performed to detect the stability of RNAs under control or PD-L1 knockdown conditions. As showed in FIG. 7g, the stability of firefly luciferase mRNA significantly decreased after PD-L1 was depleted, indicating that the ‘GVAGAW’ (where V is A, C, or G, and where W is A or U; SEQ ID NO:1) motif is important for PD-L1 binding and regulation of RNA stability.

In summary, PD-L1 not only functions extracellularly by binding with PD-1 to suppress the immune system, but also intracellularly by increasing mRNA stability of target genes. Specifically, PD-L1 regulates a number of genes involved in the DNA damage response pathway genome-widely (FIG. 7h). Because PD-L1 is overexpressed in many cancers, these results could shed new light on the role of PD-L1 in cancer pathogenesis. In addition, these findings have important translational implications, given the emergence of PD-L1 as a therapeutic target in cancer and interest in combining PD-L1 inhibition with DNA damaging therapy. Clinically available drugs that disrupt the PD1/PD-L1 interaction were not able to affect radiation sensitivity. Targeting the intracellular signaling pathway of PD-L1 as described herein can be a viable therapeutic use for inhibiting intracellular activity of PD-L1.

### Example 2: PD-L1 (B7-H1) Inhibitory Peptides

Peptides that can block the PD-L1 binding site for NBS1 RNA were designed.

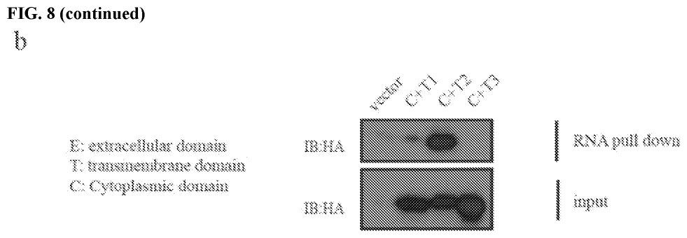

Three different truncations of the PD-L1 cytoplasmic domain were constructed. Each truncation contained 10 amino acids. Since expressing the cytoplasmic domain of PD-L1 is challenging, the transmembrane domain was included in all three truncations (labeled as C+T1, C+T2 and C+T3; FIG. 8a).

These three PD-L1 truncations were used to pull down NBS1 mRNA. It was observed that amino acids 270-279 (sequence: KKCGIQDTNS (SEQ ID NO:31)) from the PD-L1 cytoplasmic domain are required for the PD-L1/RNA interaction (FIG. 8b). Peptides are as numbered in the National Center for Biotechnology Information (NCBI) GenPept Accession No.: Q9NZQ7.1. A peptide including amino acids KKCGIQDTNS (SEQ ID NO:31) was synthesized to perform the blocking function and act as a polypeptide that inhibits (e.g., reduces or eliminates) the function of an intracellular domain of PD-L1.

To enhance cellular uptake, a CPP having the amino acid sequence RRRRRRRR (SEQ ID NO:32)) was connected to the C-terminal end of the KKCGIQDTNS (SEQ ID NO:31) polypeptide.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having cancer, said method comprising:
administering a compound that inhibits the function of an intracellular domain of PD-L1 to said mammal under conditions wherein cancer cells present are sensitized to one or more cancer treatments.

2. A method for treating a mammal having cancer, said method comprising:
administering a compound that inhibits the function of an intracellular domain of PD-L1 to said mammal; and
administering one or more cancer treatments said mammal under conditions wherein number of cancer cells present in said mammal is reduced.

3. The method of claim 1, wherein said mammal is a human.

4. The method of claim 1, where said cancer is breast cancer or colorectal cancer.

5. (canceled)

6. The method of claim 1, wherein said one or more cancer treatments are selected from the group consisting of radiation therapy, chemotherapy, hormone therapy, targeted therapy, and cytotoxic therapy.

7. The method of claim 6, wherein the one or more cancer treatments comprises radiation therapy and wherein said radiation therapy is irradiation.

8. The method of claim 6, wherein the one or more cancer treatments comprises chemotherapy and wherein said chemotherapy is cisplatin.

9. The method of claim 1, wherein the compound that inhibits the function of an intracellular domain of PD-L1 comprises one or more shRNA molecules.

10. (canceled)

11. The method of claim 9, wherein said one or more shRNA molecules comprise the sequence GACCUAUAUGUGGUAGAGUAU (SEQ ID NO:3).

12. (canceled)

13. The method of claim 9, wherein said one or more shRNA molecules comprise the sequence CGAAUUACUGUGAAAGUCAAU (SEQ ID NO:4).

14. The method of claim 1, wherein the compound that inhibits the function of an intracellular domain of PD-L1 comprises one or more polypeptides.

15. (canceled)

16. The method of claim 15, wherein said one or more polypeptides comprise the sequence KKCGIQDTNS (SEQ ID NO:31).

17. The method of claim 1, wherein said one or more polypeptides further comprises a cell penetrating peptide.

18. The method of claim 17, wherein said cell penetrating peptide comprises the sequence RRRRRRRR (SEQ ID NO:32)

19. A method for identifying a compound that inhibits the function of an intracellular PD-L1 domain, said method comprising:
contacting a candidate compound with an intracellular domain of PD-L1;
determining if said candidate compound inhibits the function of said intracellular domain of PD-L1; and
identifying said candidate compound as a compound that inhibits the function of said intracellular PD-L1 domain when the function of the intracellular PD-L1 domain is reduced or eliminated.

20. The method of claim 19, wherein said method comprises contacting said candidate compound with cell-free PD-L1.

21. The method of claim 19, wherein said method comprises contacting said candidate compound with cells expressing PD-L1.

22. The method of claim 21, wherein the PD-L1 is endogenous PD-L1.

23. The method of claim 21, wherein said PD-L1 is recombinant PD-L1.

24. The method of claim 19, wherein said determining comprises a co-immunoprecipitation or a nuclear run-on assay.

25. (canceled)
